(19) United States
(12) Patent Application Publication
(10) Pub. No.: US 2003/0054396 A1
(43) Pub. Date: Mar. 20, 2003

(54) Enzymatic light amplification

(76) Inventor: Michael P. Weiner, Guilford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/236,871

(22) Filed: Sep. 6, 2002

Related U.S. Application Data
(60) Provisional application No. 60/318,218, filed on Sep. 7, 2001.
(60) Provisional application No. 60/335,950, filed on Oct. 30, 2001.

(51) Int. Cl. C12Q 1/68; G01N 33/53; C07H 21/04; C12N 9/24; C07K 14/435

(52) U.S. Cl. 435/6; 435/200; 530/395; 536/23.1; 435/7.5

(57) ABSTRACT

Reversibly labeled nucleotides and methods involving the nucleotides are disclosed. The methods included methods of determining a sequence of a nucleic acid.

### RELATED APPLICATIONS

This Application claims the benefit of priority from U.S. Ser. No. 60/318,218 filed Sep. 7, 2001 and U.S. Ser. No. 60/335,950 filed Oct. 30, 2001. Both applications are incorporated herein by reference in their entirity.

### ABSTRACT

Disclosed herein are methods and apparatuses for sequencing a nucleic acid. In one aspect, the method includes annealing a population of circular nucleic acid molecules to a plurality of anchor primers linked to a mobile solid support, and amplifying those members of the population of circular nucleic acid molecules which anneal to the target nucleic acid, and then sequencing the amplified molecules by detecting the presence of a modified nucleotide such as biotin-S—S-dUTP.

### FIELD OF THE INVENTION

The invention relates to methods and apparatuses for determining the sequence of a nucleic acid.

### BACKGROUND OF THE INVENTION

Many diseases are associated with particular DNA sequences. The DNA sequences are often referred to as DNA sequence polymorphisms to indicate that the DNA sequence associated with a diseased state differs from the corresponding DNA sequence in non-afflicted individuals. DNA sequence polymorphisms can include, for example, insertions, deletions, or substitutions of nucleotides in one sequence relative to a second sequence. An example of a particular DNA sequence polymorphism is 5′-ATCG-3′, relative to the sequence 5′-ATGG-3′at a particular location in the human genome. The nucleotide ‘C’ in the first sequence has been replaced by the nucleotide ‘G’ in the second sequence. The first sequence is associated with a particular disease state, whereas the latter sequence is associated with individuals not suffering from the disease. Thus, the presence of the nucleotide sequence ‘5-ATCG-3’ indicates the individual has a high susceptibility to the particular disease. This particular type of sequence polymorphism is known as a single-nucleotide polymorphism, or SNP, because the sequence difference is due to a change in one nucleotide.

Techniques which enable the rapid detection of as little as a single DNA base change are therefore important methodologies for use in genetic analysis. Because the size of the human genome is large, on the order of 3 billion base pairs, techniques for identifying polymorphisms must be sensitive enough to specifically identify the sequence containing the polymorphism in a potentially large population of nucleic acids.

Typically a DNA sequence polymorphism analysis is performed by isolating DNA from an individual, manipulating the isolated DNA, for example, by digesting the DNA with restriction enzymes and/or amplifying a subset of sequences in the isolated DNA. The manipulated DNA is then examined further to determine if a particular sequence is present.

Commonly used procedures for analyzing the DNA include electrophoresis. Common applications of electrophoresis include agarose or polyacrylamide gel electrophoresis. DNA sequences are inserted, or loaded, on the gels and subjected to an electric field. Because DNA carries a uniform negative charge, DNA will migrate through the gel at a speed that is inversely proportional to its size—the migration rate is affected by properties including sequence length, three-dimensional conformation and interactions with the gel matrix ratio upon application of the electrical field). Thus, in most DNA analysis methods, smaller DNA molecules will migrate more rapidly through the gel than larger fragments. Electrophoresis is usually continued for a sufficient length of time for the optimal separation of the DNA fragments of interest. After separation, DNA molecules can be detected using a variety of detection methodologies. For some applications, particular DNA sequences are identified by the presence of detectable tags, such as radioactive labels, attached to specific DNA molecules. Other techniques include, for example, Southern blots, RNA blots, fluorescent dyes, and the like.

Electrophoretic-based separation analyses can be less desirable for applications in which it is desirable to rapidly, economically, and accurately analyze a large number of nucleic acid samples for particular sequence polymorphisms. For example, electrophoreses-based analysis can require a large amount of input DNA. In addition, processing the large number of samples required for electrophoretic-based nucleic acid based analyses can be labor intensive. Furthermore, these techniques can require samples of identical DNA molecules, which must be created prior to electrophoresis at costs that can be considerable. At this time, the high cost and labor associated with current screening methods have prevented widespread adoption of genetic screening for diagnosis.

Recently, automated electrophoresis systems have become available. However, the throughput and cost of current automated electrophoresis is still too high for widespread adoption. Thus, the need for non-electrophoretic methods for sequencing is great.

Several alternatives to electrophoretic-based sequencing have been described. These include scanning tunnel electron microscopy, sequencing by hybridization, and single molecule detection methods.

Another alternative to electrophoretic-based separation is solid substrate-based nucleic acid analyses. These methods typically rely upon the use of large numbers of nucleic acid probes affixed to different locations on a solid support. These solid supports can include, for example, glass surfaces, plastic microtiter plates, plastic sheets, thin polymers, or semi-conductors. The probes can be, for example, adsorbed or covalently attached to the support, or can be microencapsulated or otherwise entrapped within a substrate membrane or film.

Substrate-based nucleic acid analyses can include applying a sample nucleic acid known or suspected of containing a particular sequence polymorphism to an array of probes attached to the solid substrate. The nucleic acids in the population are allowed to hybridize to complementary sequences attached to the substrate, if present. Hybridizing nucleic acid sequences are then detected in a detection step.

Solid support matrix-based hybridization and sequencing methodologies can require a high sample-DNA concentration and can be hampered by the relatively slow hybridization kinetics of nucleic acid samples with immobilized oligonucleotide probes. Often, only a small amount of template DNA is available, and it can be desirable to have high concentrations of the target nucleic acid sequence. Thus, substrate based detection analyses often include a step in which copies of the target nucleic acid, or a subset of sequences in the target nucleic acid, is amplified. Methods based on the Polymerase Chain Reaction (PCR), for example, can increase a small number of probes targets by several orders of magnitude in solution. However, PCR can be difficult to incorporate into a solid-phase approach because the amplified DNA is not immobilized onto the surface of the solid support matrix.

Solid-phase based detection of sequence polymorphisms has been described. An example is a “mini-sequencing” protocol based upon a solid phase principle described by Hultman, et al., 1988. Nucl. Acid. Res. 17: 4937-4946; Syvanen, et al.,1990. Genomics 8: 684-692). In this study, the incorporation of a radiolabeled nucleotide was measured and used for analysis of a three-allelic polymorphism of the human apolipoprotein E gene. However, such radioactive methods are not well-suited for routine clinical applications, and hence the development of a simple, highly sensitive non-radioactive method for rapid DNA sequence analysis has also been of great interest.

### SUMMARY OF THE INVENTION

The invention is based in part on the discovery of a highly sensitive method for determining the sequences of nucleic acids attached to solid substrates.

Accordingly, in one aspect, the invention includes a substrate for analyzing a nucleic acid. The substrate includes a fiber optic surface onto which has been affixed one or more nucleic acid sequences. The fiber optic surface can be cavitated, for example, a hemispherical etching of the opening of a fiber optic. The substrate can in addition include a plurality of bundled fiber optic surfaces, where one or more of the surfaces have anchored primers. The substrate for analyzing a nucleic acid can also include either a flat or micro-machined surfaces. Mobile solid supports, for example beads of various compositions, for example glass or latex, can be attached, embedded and or deposited on or in any of the aforementioned surfaces.

In another aspect, the invention includes an apparatus for analyzing a nucleic acid sequence. The apparatus can include a reagent delivery chamber, for example, a perfusion chamber, wherein the chamber includes a nucleic acid substrate, a conduit in communication with the perfusion chamber, an imaging system, for example, a fiber optic system, in communication with the perfusion chamber; and a data collection system in communication with the imaging system. The substrate can be a planar substrate. In other embodiments, the substrate can be the afore-mentioned fiber optic surface having nucleic acid sequences affixed to its termini, a mobile solid support deposited in or on the aforementioned fiber optic support, or a treated surface either on or within to which a nucleic acid is attached.

In a further aspect, the invention includes a method for sequencing a nucleic acid. The method include providing a primed anchor primer circular template complex and combining the complex with a polymerase, and nucleotides to generate concatenated, linear complementary copies of the circular template. The extended anchor primer-circular template complex can be generated in solution and then linked to a solid substrate. Alternatively, one or more nucleic acid anchor primers can be linked to a solid or mobile-solid support and then annealed to a plurality of circular nucleic acid templates. The linked nucleic acid anchor primer is then annealed to a single-stranded circular template to yield a primed anchor primer-circular template complex.

A sequencing primer is annealed to the circular nucleic acid template to yield a primed sequencing primer-circular nucleic acid template complex. Annealing of the sequencing primer can occur prior to, or after, attachment of the extended anchor primer to the solid substrate. The sequence primer is then extended with a polymerase and a predetermined modified nucleotide triphosphate which is reversibly labeled to yield a sequencing product. If the predetermined modified nucleotide is incorporated into the primer, then the label may be detected by an enzymatic signal amplification. If the predetermined nucleotide is incorporated in the sequencing primer multiple times, for example, the concatenated nucleic acid template has multiple identical nucleotides, the quantity or concentration of the amplification signal is measured to determine the number of nucleotides incorporated. If desired, additional predetermined nucleotide triphosphates can be added, for example, sequentially, and the presence or absence of nucleotide additions associated with each reaction can be determined.

In a still further aspect, the invention includes a method for sequencing a nucleic acid by providing one or more nucleic acid anchor primers linked to a plurality of anchor primers linked to a fiber optic surface substrate, for example, the solid substrate discussed above.

In various embodiments of the apparatuses and methods described herein, the solid substrate includes two or more anchoring primers separated by approximately 10 μm to approximately 200 μm, 50 μm to approximately 150 μm, 100 μm to approximately 150 μm, or 150 μm. The solid support matrix can include a plurality of pads that are covalently linked to the solid support. The surface area of the pads can be, for example, 10 μm² and one or more pads can be separated from one another by a distance ranging from approximately 50 μm to approximately 150 μm.

In preferred embodiments, at least a portion of the circular nucleic acid template is single-stranded DNA. The circular nucleic acid template can be, for example, genomic DNA or RNA, or a cDNA copy thereof. The circular nucleic acid can be, for example, 10-10,000 or 10-1000, 10-200, 10-100, 10-50, or 20-40 nucleotides in length.

In some embodiments, multiple copies of one or more circular nucleic acids in the population are generated by a polymerase chain reaction. In other embodiments, the primed circular template is extended by tandem amplification (TA) to yield a single-stranded concatamer of the annealed circular nucleic acid template. If desired, the template amplified by tandem amplification and be further amplified by annealing a reverse primer to the single-stranded concatamer to yield a primed concatamer template and combining the primed concatamer template with a polymerase enzyme to generate multiple copies of the concatamer template. In still further embodiments, the template can be extended by a combination of PCR and tandem-amplification.

In preferred embodiments, the nucleotide is modified to contain a disulfide-derivative of a hapten such as biotin. The addition of the modified nucleotide to the nascent primer annealed to the anchored substrate is analyzed by a post-polymerization step that includes i) sequentially binding of, in the example where the modification is a biotin, an avidin- or streptavidin-conjugated moiety linked to an enzyme molecule, ii) the washing away of excess avidin- or streptavidin-linked enzyme, iii) the flow of a suitable enzyme substrate under conditions amenable to enzyme activity, and iv) the detection of enzyme substrate reaction product or products.

A preferred enzyme for detecting the hapten is horse-radish peroxidase. If desired, a wash buffer, can be used between addition of various reactants herein. Apyrase can be used to remove unreacted dNTP used to extend the sequencing primer. The wash buffer can optionally include apyrase.

Example haptens, for example, biotin, digoxygenin, the fluorescent dye molecules cy3 and cy5, and fluorescein, are incorporated at various efficiencies into extended DNA molecules. The attachment of the hapten can occur through linkages via the sugar, the base, and via the phosphate moiety on the nucleotide. Example means for signal amplification include fluorescent, electrochemical and enzymatic. In a preferred embodiment using enzymatic amplification, the enzyme, for example alkaline phosphatase (AP), horse-radish peroxidase (HRP), β-galactosidase, luciferase, can include those for which light-generating substrates are known, and the means for detection of these light-generating (chemiluminescent) substrates can include a CCD camera.

In a preferred mode, the modified base is added, detection occurs, and the hapten-conjugated moiety is removed or inactivated by use of either a cleaving or inactivating agent. For example, if the cleavable-linker is a disulfide, then the cleaving agent can be a reducing agent, for example dithiothreitol (DTT), β-mercapthoethanol, etc. Other embodiments of inactivation include heat, cold, chemical denaturants, surfactants, hydrophobic reagents, and suicide inhibitors.

The anchor primer sequence can include a group that can link the anchor primer to the solid support via a group attached to the solid support. In some embodiments, the anchor primer is conjugated to a biotin-bovine serum albumin (BSA) moiety. The biotin-BSA moiety can be linked to an avidin-biotin group on the solid support. If desired, the biotin-BSA moiety on the anchor primer can be linked to a BSA group on the solid support in the presence of silane.

In some embodiments, the solid support includes at least one optical fiber.

The invention also provides a method for profiling the concentrations of mRNA transcripts present in a cell. The identity of a transcript may be determined by the sequence at its 3′ terminus (additional fragments may be used to distinguish between splice variants with identical 3′ sequence). A sequencing apparatus having 10,000 sites could, in a single run, determine the mRNA species present at a concentration of 1:10,000 or higher. Multiple runs, or multiple devices, could readily extend the limit to 1:100,000 or 1:1,000,000. This performance would be superior to current technologies, such as microarray hybridization, which have detection limits in the range 1:10,000 to 1:100,000.

In a further embodiment, the sequence of the amplified nucleic acid can be determined using by-products of RNA synthesis. In this embodiment, an RNA transcript is generated from a promoter sequence present in the circular nucleic acid template library. Suitable promoter sites and their cognate RNA polymerases include RNA polymerases from E. coli, the RNA polymerase from the bacteriophage T₃, the RNA polymerase from the bacteriophage T₇, the RNA polymerase from the bacteriophage SP6, and the RNA polymerases from the viral families of bromoviruses, tobamoviruses, tombusvirus, lentiviruses, hepatitis C-like viruses, and picornaviruses. To determine the sequence of an RNA transcript, a predetermined modified NTP, i.e., an ATP, CTP, GTP, or UTP, is incubated with the template in the presence of the RNA polymerase. Incorporation of the modified NTP into a nascent RNA strand can be determined by assaying for the presence of the modification using the enzymatic detection discussed herein.

The disclosures of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. Unless expressly stated otherwise, the techniques employed or contemplated herein are standard methodologies well known to one of ordinary skill in the art. The examples of embodiments are for illustration purposes only. All patents and publications cited in this specification are incorporated by reference.

An embodiment of the invention is directed to reversibly labeled nucleotides. The reversible nucleotides contains three parts in its most basic structure. The first part is a nucleotide or nucleoside. The second part is a detectable label with a light generating moiety. The light generating moiety may generate light (i.e., photons) on its own or it may be an enzyme that participate or promotes a chemical reaction which generates light. In the case of an enzyme, contacting the enzyme with the appropriate substrate will allow light to be generated.

The third part is a linker that connects the first two parts. The linker may be a chain with a mostly carbon backbone of between 10 and 30 carbons, preferably between 10 and 24 carbons, more preferably between 18 and 22 carbons, most preferably between 19 and 21 carbons such as, for example 20 carbons. The carbon backbone may be linear or branched but in this description only the carbon in the backbone is counted. Furthermore, while carbon backbone is preferred, other molecules such as nitrogen and sulfur (See FIG. 2) may be used instead of carbon. It is understood that the carbon molecules may be contiguous or noncontiguous.

The linker may for example, contain a cleavable group, such as, for example, a disulfide group in the middle of the linker chain. For example, a carbon chain of 10 carbons, connected to two sulfurs in a disulfide bone, and further connected to an additional 10 carbons is also a linker of the invention.

The nucleotide is reversibly labeled because the label or labeling function may be removed or inactivated. For example, in the structure described above, a cleavage of the cleavage group in the linker would separate the light generating moiety from the nucleotide. The labeling may also be reversed by inactivating the light generating moiety. Inactivation may be performed, for example, by photobleaching.

The reversible labeled nucleotide may be a nucleotide monophosphate, a nucleotide diphosphate, and a nucleotide triphosphate. Sometimes, these molecules are also called nucleoside monophosphate, a nucleoside diphosphate, and a nucleoside triphosphate respectively. Examples of such nucleotides include dATP, dTTP, dGTP, dCTP, ATP, UTP, GTP and CTP.

In one embodiment, the light generating moiety is directly connected to the linker. For example, fluorescent dye molecule, fluorescein or a combination of these two molecules may be connected to the linker by a specific binding pair. Examples of specific binding pairs may be, biotin/avidin, biotin/streptavidin, or functional derivatives, analogs and genetically engineered versions of these molecules. An example of such a derivative is the disulfide derivative of biotin or digoxygenin. Another example of a derivative is a reduced affinity avidin or streptavidin (See, e.g., Pierce catalog or U.S. Pat. No. 6,207,390). Other examples of binding pairs include antigen/antibody, hapten/peptide, maltose/maltose binding protein, protein A/antibody fragment, protein G/antibody fragment, polyhistidine/nickel, glutathione S transferase/glutathione and derivatives, functional fragments, and functional analogs thereof. Other examples of light generating moiety include thereof.

The light generating moiety may be any enzyme or chemical that generates light (photons) directly or indirectly. Examples of such chemicals and enzymes include alkaline phosphatase, horse radish peroxidase, green fluorescent protein, blue fluorescent protein, red fluorescent protein, beta-galactosidase, chloramphenicol acetyltransferase, beta-glucoronidase, luciferases, b-lactamase blue EBFP, cyan ECFP, yellow-green EYFP, destabilized GFP variants, stabilized GFP amd variants and fusion variants and derivatives of these proteins. More preferably, the light generating moiety is alkaline phosphatase or horse radish peroxidase.

Methods of detecting the light generating moieties are known. Generally, light is produced by contacting these moieties with commercially available substrats. Specific substrates include ATP, NBR/BCIP, ascorbate, ferrocyanide, cytochrome C. X-gal, Acetyl CoA, n-butytyl CoA, chloramphenicol, glucoronides, antidigoxigenin-POD, diaminobenzidine, luciferin, beta-lactam, glucororides, H₂O₂ or a combination of these reagents. Alternatively, the label may emit light on its own or be a fluorescent label capable of emitting light when light the correct wavelength is provided as an excitation source. Examples of these labels include fluorescent dye molecules, and fluorescein.

It is preferred that the detectable labels of the invention should be detectable by using chemical or enzymatic methods.

The linker may be connected to the nucleotides by connecting to a sugar, a base or a phosphate moiety on said nucleotide triphosphate. In a preferred embodiment, the linker is connected to the nucleotide triphosphate by a cleavable bond.

The cleavable bond may be a covalent or ionic bond. An example of a covalent bond is a disulfide bond (S—S) which can be cleaved by exposure to a reducing agent such as dithiothreitol and β-mercaptoethanol. The cleavable bond may also be cleaved by exposure to heat, cold, chemical denaturants, surfactants, hydrophobic reagents, and suicide inhibitors.

The general structure of the reversibly labeled nucleotide is described above with the methods of detecting and cleaving or inactivating the labeled. These structures and methods are applicable for all the methods of the invention so that any of the structure above may be used in any of the methods of the invention.

Another embodiment of the invention is directed to a method of determining the incorporation of a nucleotide into an elongating chain of a nucleic acid. In the first step, a nucleic acid is first contacted with a first species of a reversibly labeled nucleotide triphosphate like the NTPs and dNTPs described above. In the second step the incorporation of the first species may be monitored by detecting light emitted by said light generating moiety. The light emitted may be analyzed by known techniques such as measuring light intensity, light duration, or total photons emitted for a period of time, (a function of intensity and duration). If the detectable label is known to emit light of a certain wavelength, the detection may be tailored for that wave length. One way of tailoring the light detection wavelength is by the use of a filter.

In addition, the following optional third step of inactivating or detaching the label may be performed. Methods of inactivating the label, such as by photobleaching are discussed in another section. The label may be detached in a number of ways. The cleavable bond may be cleaved or if the nucleotide comprises a binding pair, it may be cleaved. Second, if the binding pair is used, it may also be cleaved. Methods of cleaving the binding pair is known. For example, if the binding pair uses a reduced affinity or heat labile streptavidin, the binding pair may be separated by heat or by the addition of a molecular excess of avidin.

The optional fourth step merely requires the repeated performance of the first, second and third steps with a separate reversibly labeled nucleotide triphosphate that is different that the previous nucleotide triphosphate used.

Using the method above, the sequence of the elongating nucleic acid may be determined by recording the order of nucleotide triphosphate used in the first step and the results of the second step. For example, if dATP is used in the first step and 1 dATP was incorporated by measuring the light emitted in the second step, then it is known that the added base is an “A.” step (b).

The light generating moiety of the invention may be alkaline phosphatase, horse radish peroxidase, digoxygenin, fluorescent dye molecule, or fluorescein. Other light generating moieties include green fluorescent protein, blue fluorescent protein, red fluorescent protein, beta-galactosidase, chloramphenicol acetyltransferase, beta-glucoronidase, luciferases, b-lactamase and derivatives thereof.

The detection substrate, may be ATP, NBR/BCIP, ascorbate, ferrocyanide, cytochrome C. X-gal, Acetyl CoA, n-butytyl CoA, chloramphenicol, glucoronides, antidigoxigenin-POD, diaminobenzidine, luciferin, beta-lactam, glucororides, H₂O₂ or a combination of these reagents.

Another embodiment of the invention is directed to determining the sequence of a template nucleic acid. The template nucleic acid is used as a template for the elongation of a second nucleic acid using the method of the invention. Since the sequence of the elongated nucleic acid may be known using the methods of the invention, the sequence of the template nucleic acid may be deduced by deducing the complement of the elongating nucleic acid.

The methods disclosed may be used to monitor elongation or to sequence a template nucleic acid under different chain elongation conditions such as during a transcription reaction, during a nucleic acid replication reaction, or during a reverse transcription reaction.

The light emitted by the detectable label using the method of the invention may be non-stoichiometric. That is, the incorporation of one detectable base results in the emission of more than one photon at the detection stage. While the methods work if only one photon is emitted per detectable label, it is preferred that at least 10 photons are emitted per detectable label incorporated into the nucleic acid undergoing elongation. More preferably, at least 20 photons are emitted. Even more preferably, at least 100 photons are emitted. Most preferably over 1000 photons are emitted.

Another embodiment of the invention is directed to a method of sequencing a nucleic acid, the method. In the method, a nucleic acid anchor primer is provided. Then a plurality of single-stranded nucleic acid templates disposed within a plurality of cavities on a planar surface is provided. Each cavity forms an analyte reaction chamber and each reaction chambers have a center to center spacing of between 5 to 200 μm. Then effective amount of the nucleic acid anchor primer is annealed to at least one of the single-stranded templates to yield a primed anchor primer-template complex. Then, the primed anchor primer-template complex is combined with a polymerase to form an extended anchor primer covalently linked to multiple copies of a nucleic acid complementary to the nucleic acid template. Next, an effective amount of a sequencing primer is annealed to one or more copies of the covalently linked complementary nucleic acid and the sequencing primer with a polymerase and a predetermined nucleotide triphosphate is extended to yield a sequencing product. The nucleotide triphosphate used is a reversibly labeled nucleotide triphosphate discussed supra. By detecting the amount of incorporation of the reversible labeled triphosphate the sequence of the nucleic acid may be determined. In using the reversibly labeled nucleotide triphosphate in this method, the NTP may be conjugated to only one half of the binding pair initially. During the detection step, the detectable label connected to the complementary half of the binding pair is added. The binding pair is allowed to form. Thus, the label will be incorporated into the elongated nucleic acid. Then the label may be detected normally.

The label used may be any label described previously such as biotin, digoxygenin, fluorescent dye molecule, fluorescein and derivatives and combinations thereof. The fluorescent dye molecule may be cy3 or cy5. The label and linker may be connected to a sugar, a base or a phosphate moiety on the nucleotide triphosphate. Furthermore, the label may be connected to the nucleotide triphosphate by a cleavable bond. The label may be connected to the nucleotide by both a cleavable bond and a binding pair. The cleavable bond may be covalent (S—S) or ionic bond (avidin biotin or streptavidin biotin). The cleavable bond may be cleaved by a reducing agent such as, for example, dithiothreitol or β-mercaptoethanol. Other methods of cleavage may include exposure to heat, cold, chemical denaturants, surfactants, hydrophobic reagents, and suicide inhibitors.

Alternatively, the label may be inactivated by exposure to heat, cold, chemical denaturants, surfactants, hydrophobic reagents, and suicide inhibitors. For example, heat labile streptavidin is sensitive to heat. However, the methods of the invention may be perform under heat if thermophilic polymerase is used.

The nucleic acid to be sequenced may be circular or single stranded circular. It could contain at least 100 copies of a nucleic acid sequence, each copy covalently linked end to end.

The reaction chamber may have a width in at least one dimension of between 0.3 μm and 100 μm, preferably between 0.3 μm and 20 μm, more preferably between 0.3 μm and 10 μm. One example would be between 20 μm and 70 μm.

The number of cavities may number greater than 400,000. Preferably it is between 400,000 and 20,000,000, more preferably it is between 1,000,000 and 16,000,000.

The center to center spacing of the reaction chamber/cavities may be between 10 to 150 μm, such as between 50 to 100 μm or between 10 μm and 100 μm.

Each cavity may have a depth that is between 0.25 and 5 times the size of the width of the cavity such as between 0.3 and 1 times the size of the width of the cavity.

The methods of the invention may be applied to a nucleic acid that is further amplified to produce multiple copies of the nucleic acid sequence after being disposed in the reaction chamber. Amplification techniques include polymerase chain reaction, ligase chain reaction and isothermal DNA amplification.

Finally, the nucleic acid may be immobilized in the reaction chamber.

### BRIEF DESCRIPTION OF THE DRAWINGS

FIG. 1 depicts the specifics of enzymatic sequencing reaction.

FIG. 2 depicts the synthesis of biotin-S—S-dUTP nucleotide.

FIG. 3 depicts the structure of biotin-conjugated nucleotide triphosphates.

FIG. 4 depicts the enzymatic sequencing reaction.

FIG. 5 depicts the acridan-based substrates.

FIG. 6 depicts the luminol-based substrates.

FIG. 7 depicts the dioxetane based substrates.

FIG. 8 depicts the enzyme activity on various substrates.

FIG. 9 depicts the enzymatic sequencing reaction.

### DETAILED DESCRIPTION OF THE INVENTION

The methods and apparatuses described herein allow for the determination of nucleic acid sequence information without the need for first cloning a nucleic acid. In addition, the method is highly sensitive and can be used to determine the nucleotide sequence of a template nucleic acid, which is present in only a few copies in a starting population of nucleic acids. Further, the method can be used to determine simultaneously the sequences of a large number of nucleic acids.

The methods and apparatuses described are generally useful for any application in which the identification of any particular nucleic acid sequence is desired. For example, the methods allow for identification of single nucleotide polymorphisms (SNPs), haplotypes involving multiple SNPs or other polymorphisms on a single chromosome, and transcript profiling. Other uses include sequencing of artificial DNA constructs to confirm or elicit their primary sequence, or to identify specific mutant clones from random mutagenesis screens, as well as to obtain the sequence of cDNA from single cells, whole tissues or organisms from any developmental stage or environmental circumstance in order to determine the gene expression profile from that specimen. In addition, the methods allow for the sequencing of PCR products and/or cloned DNA fragments of any size isolated from any source.

The methods described herein include a sample preparation process that results in a solid or a mobile solid substrate array containing a plurality of anchor primers covalently linked to a nucleic acid containing one or more copies complementary to a target nucleic acid. Formation of the covalently linked anchor primer and one or more copies of the target nucleic acid preferably occurs by annealing the anchor primer to a complementary region of a circular nucleic acid, and then extending the annealed anchor primer with a polymerase to result in formation of a nucleic acid containing one or more copies of a sequence complementary to the circular nucleic acid.

Attachment of the anchor primer to a solid or mobile solid substrate can occur before, during, or subsequent to extension of the annealed anchor primer. Thus, in one embodiment, one or more anchor primers are linked to the solid or a mobile solid substrate, after which the anchor primer is annealed to a target nucleic acid and extended in the presence of a polymerase. Alternatively, in a second embodiment, an anchor primer is first annealed to a target nucleic acid, and a 3′OH terminus of the annealed anchor primer is extended with a polymerase. The extended anchor primer is then linked to the solid or mobile solid substrate. By varying the sequence of anchor primers, it is possible to specifically amplify distinct target nucleic acids present in a population of nucleic acids.

Sequences in the target nucleic acid can be identified in a number of ways. Preferably, a sequencing primer is annealed to the amplified nucleic acid and used to generate a sequencing product. The nucleotide sequence of the sequence product is then determined, thereby allowing for the determination of the nucleic acid. Similarly, in one embodiment, the template nucleic acid is amplified prior to its attachment to the bead or other mobile solid support. In other embodiments, the template nucleic acid is attached to the bead prior to its amplification.

The methods of the present invention can be also used for the sequencing of DNA fragments generated by analytical techniques that probe higher order DNA structure by their differential sensitivity to enzymes, radiation or chemical treatment (e.g., partial DNase treatment of chromatin), or for the determination of the methylation status of DNA by comparing sequence generated from a given tissue with or without prior treatment with chemicals that convert methyl-cytosine to thymidine (or other nucleotide) as the effective base recognized by the polymerase. Further, the methods of the present invention can be used to assay cellular physiology changes occurring during development or senescence at the level of primary sequence.

I. Apparatus for Sequencing Nucleic Acids

This invention provides an apparatus for sequencing nucleic acids, which generally comprises one or more reaction chambers for conducting a sequencing reaction, means for delivering reactants to and from the reaction chamber(s), and means for detecting a sequencing reaction event. In another embodiment, the apparatus includes a reagent delivery cuvette containing a plurality of cavities on a planar surface. In a preferred embodiment, the apparatus is connected to at least one computer for controlling the individual components of the apparatus and for storing and/or analyzing the information obtained from detection of the sequence reaction event.

The invention also provides one or more reaction chambers are arranged in the form of an array on an inert substrate material, also referred to herein as a “solid support”, that allows for combination of the reactants in a sequencing reaction in a defined space and for detection of the sequencing reaction event. Thus, as used herein, the terms “reaction chamber” or “analyte reaction chamber” refer to a localized area on the substrate material that facilitates interaction of reactants, e.g., in a nucleic acid sequencing reaction. As discussed more fully below, the sequencing reactions contemplated by the invention preferably occur on numerous individual nucleic acid samples in tandem, in particular simultaneously sequencing numerous nucleic acid samples derived from genomic and chromosomal DNA. The apparatus of the invention therefore preferably comprises an array having a sufficient number of reaction chambers to carry out such numerous individual sequencing reactions. In one embodiment, the array comprises at least 1,000 reaction chambers. In another embodiment, the array comprises greater than 400,000 reaction chambers, preferably between 400,000 and 20,000,000 reaction chambers. In a more preferred embodiment, the array comprises between 1,000,000 and 16,000,000 reaction chambers.

The reaction chambers on the array typically take the form of a cavity or well in the substrate material, having a width and depth, into which reactants can be deposited. One or more of the reactants typically are bound to the substrate material in the reaction chamber and the remainder of the reactants are in a medium which facilitates the reaction and which flows through the reaction chamber. When formed as cavities or wells, the chambers are preferably of sufficient dimension and order to allow for (i) the introduction of the necessary reactants into the chambers, (ii) reactions to take place within the chamber and (iii) inhibition of mixing of reactants between chambers. The shape of the well or cavity is preferably circular or cylindrical, but can be multisided so as to approximate a circular or cylindrical shape. In another embodiment, the shape of the well or cavity is substantially hexagonal. The cavity can have a smooth wall surface. In an additional embodiment, the cavity can have at least one irregular wall surface. The cavities can have a planar bottom or a concave bottom. The reaction chambers can be spaced between 5 μm and 200 μm apart. Spacing is determined by measuring the center-to-center distance between two adjacent reaction chambers. Typically, the reaction chambers can be spaced between 10 μm and 150 μm apart, preferably between 50 μm and 100 μm apart. In one embodiment, the reaction chambers have a width in one dimension of between 0.3 μm and 100 μm. The reaction chambers can have a width in one dimension of between 0.3 μm and 20 μm, preferably between 0.3 μm and 10 μm, more preferably between 20 μm and 70 μm and most preferably about 6 μm. Ultimately the width of the chamber could be dependant on whether the nucleic acid samples require amplification. If no amplification is necessary, then smaller, e.g., 0.3 μm is preferred. If amplification is necessary, then larger, e.g., 6 μm is preferred. The depth of the reaction chambers are preferably between 10 μm and 100 μm. Alternatively, the reaction chambers may have a depth that is between 0.25 and 5 times the width in one dimension of the reaction chamber or, in another embodiment, between 0.3 and 1 times the width in one dimension of the reaction chamber.

In another aspect, the invention involves an apparatus for determining the nucleic acid sequence in a template nucleic acid polymer. The apparatus includes an array having a plurality of cavities on a planar surface. Each cavity forms an analyte reaction chamber, wherein the reaction chambers have a center-to-center spacing of between 5 to 200 μm. It also includes a nucleic acid delivery means for introducing a template nucleic acid polymers into the reaction chambers; and a nucleic acid delivery means to deliver reagents to the reaction chambers to create a polymerization environment in which the nucleic acid polymers will act as a template polymers for the synthesis of complementary nucleic acid polymers when nucleotides are added. The apparatus also includes a reagent delivery means for successively providing to the polymerization environment a series of feedstocks, each feedstock comprising a reversibly labeled and optionally reversibly terminated nucleotide selected from among the nucleotides from which the complementary nucleic acid polymer will be formed, such that if the nucleotide in the feedstock is complementary to the next nucleotide in the template polymer to be sequenced the nucleotide will be incorporated into the complementary polymer along with the reversible label. It also includes a detection means for detecting the label enzymatically; and a data processing means to determine the identity of each nucleotide in the complementary polymers and thus the sequence of the template polymers.

In another aspect, the invention involves an apparatus for determining the base sequence of a plurality of nucleotides on an array. The apparatus includes a reagent cuvette containing a plurality of cavities on a planar surface. Each cavity forms an analyte reaction chamber, wherein the reaction chambers have a center-to-center spacing of between 5 to 200 μm. The apparatus also includes a reagent delivery means for adding an a reversibly labeled and optionally reversibly terminated nucleotide 5′-triphosphate precursor of one known nitrogenous base to a reaction mixture in each reaction chamber. Each reaction mixture has a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the a reversibly labeled and optionally reversibly terminated nucleoside 5′-triphosphate precursor onto the 3′-end of the primer strands, provided the nitrogenous base of the a reversibly labeled and optionally reversibly terminated nucleoside 5′-triphosphate precursor is complementary to the nitrogenous base of the unpaired nucleotide residue of the templates. The apparatus also includes a detection means for detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor. The apparatus also includes a means for sequentially repeating the second and third steps wherein each sequential repetition adds and, detects the incorporation of one type of a reversibly labeled and optionally reversibly terminated nucleoside 5′-triphosphate precursor of known nitrogenous base composition. The apparatus also includes a data processing means for determining the base sequence of the unpaired nucleotide residues of the template in each reaction chamber from the sequence of incorporation of the nucleoside precursors.

II. Solid Support Material

Any material can be used as the solid support material, as long as the surface allows for stable attachment of the primers and detection of nucleic acid sequences. The solid support material can be planar or can be cavitated, e.g., in a cavitated terminus of a fiber optic or in a microwell etched, molded, or otherwise micromachined into the planar surface, e.g. using techniques commonly used in the construction of microelectromechanical systems. See e.g., Rai-Choudhury, Handbook of Microlithography, Micromachining, and Microfabrication, Volume I: Microlithography, Volume PM39, SPIE Press (1997); Madou, CRC Press (1997), Aoki, Biotech. Histochem. 67: 98-9 (1992); Kane et al., Biomaterials. 20: 2363-76 (1999); Deng et al., Anal. Chem. 72:3176-80 (2000); Zhu et al., Nat. Genet. 26:283-9 (2000). In some embodiments, the solid support is optically transparent, e.g., glass.

An array of attachment sites on an optically transparent solid support can be constructed using lithographic techniques commonly used in the construction of electronic integrated circuits as described in, e.g., techniques for attachment described in U.S. Pat. Nos. 5,143,854, 5,445,934, 5,744,305, and 5,800,992; Chee et al., Science 274: 610-614 (1996); Fodor et al., Nature 364: 555-556 (1993); Fodor et al., Science 251: 767-773 (1991); Gushin, et al., Anal. Biochem. 250: 203-211 (1997); Kinosita et al., Cell 93: 21-24 (1998); Kato-Yamada et al., J. Biol. Chem. 273: 19375-19377 (1998); and Yasuda et al., Cell 93: 1117-1124 (1998). Photolithography and electron beam lithography sensitize the solid support or substrate with a linking group that allows attachment of a modified biomolecule (e.g., proteins or nucleic acids). See e.g., Service, Science 283: 27-28 (1999); Rai-Choudhury, Handbook of Microlithography, Micromachining, and Microfabrication, Volume I: Microlithography, Volume PM39, SPIE Press (1997). Alternatively, an array of sensitized sites can be generated using thin-film technology as described in Zasadzinski et al., Science 263: 1726-1733 (1994).

III. Fiber Optic Substrate Arrays

The substrate material is preferably made of a material that facilitates detection of the reaction event. For example, in a typical sequencing reaction, binding of a dNTP to a sample nucleic acid to be sequenced can be monitored by detection of photons generated by enzyme action on phosphate liberated in the sequencing reaction. Thus, having the substrate material made of a transparent or light conductive material facilitates detection of the photons.

In some embodiments, the solid support can be coupled to a bundle of optical fibers that are used to detect and transmit the light product. The total number of optical fibers within the bundle may be varied so as to match the number of individual reaction chambers in the array utilized in the sequencing reaction. The number of optical fibers incorporated into the bundle is designed to match the resolution of a detection device so as to allow 1:1 imaging. The overall sizes of the bundles are chosen so as to optimize the usable area of the detection device while maintaining desirable reagent (flow) characteristics in the reaction chamber. Thus, for a 4096 by 4096 pixel CCD (charge-coupled device) array with 15 μm pixels, the fiber bundle is chosen to be approximately 60 mm×60 mm or to have a diameter of approximately 90 mm. The desired number of optical fibers are initially fused into a bundle or optical fiber array, the terminus of which can then be cut and polished so as to form a “wafer” of the required thickness (e.g., 1.5 mm). The resulting optical fiber wafers possess similar handling properties to that of a plane of glass. The individual fibers can be any size diameter (e.g., 3 μm to 100 μm).

In some embodiments two fiber optic bundles are used: a first bundle is attached directly to the detection device (also referred to herein as the fiber bundle or connector) and a second bundle is used as the reaction chamber substrate (the wafer or substrate). In this case the two are placed in direct contact, optionally with the use of optical coupling fluid, in order to image the reaction centers onto the detection device. If a CCD is used as the detection device, the wafer could be slightly larger in order to maximize the use of the CCD area, or slightly smaller in order to match the format of a typical microscope slide—25 mm×75 mm. The diameters of the individual fibers within the bundles are chosen so as to maximize the probability that a single reaction will be imaged onto a single pixel in the detection device, within the constraints of the state of the art. Exemplary diameters are 6-8 μm for the fiber bundle and 6-50 μm for the wafer, though any diameter in the range 3-100 μm can be used. Fiber bundles can be obtained commercially from CCD camera manufacturers. For example, the wafer can be obtained from Incom, Inc. (Charlton, Mass.) and cut and polished from a large fusion of fiber optics, typically being 2 mm thick, though possibly being 0.5 to 5 mm thick. The wafer has handling properties similar to a pane of glass or a glass microscope slide.

Reaction chambers can be formed in the substrate made from fiber optic material. The surface of the optical fiber is cavitated by treating the termini of a bundle of fibers, e.g., with acid, to form an indentation in the fiber optic material. Thus, in one embodiment cavities are formed from a fiber optic bundle, preferably cavities can be formed by etching one end of the fiber optic bundle. Each cavitated surface can form a reaction chamber. Such arrays are referred to herein as fiber optic reactor arrays or FORA. The indentation ranges in depth from approximately one-half the diameter of an individual optical fiber up to two to three times the diameter of the fiber. Cavities can be introduced into the termini of the fibers by placing one side of the optical fiber wafer into an acid bath for a variable amount of time. The amount of time can vary depending upon the overall depth of the reaction cavity desired (see e.g., Walt, et al., 1996. Anal. Chem. 70: 1888). A wide channel cavity can have uniform flow velocity dimensions of approximately 14 mm×43 mm. Thus, with this approximate dimension and at approximately 4.82×10⁻⁴ cavities/um² density, the apparatus can have approximately 290,000 fluidically accessible cavities. Several methods are known in the art for attaching molecules (and detecting the attached molecules) in the cavities etched in the ends of fiber optic bundles. See, e.g., Michael, et al., Anal. Chem. 70: 1242-1248 (1998); Ferguson, et al., Nature Biotechnology 14: 1681-1684 (1996); Healey and Walt, Anal. Chem. 69: 2213-2216 (1997). A pattern of reactive sites can also be created in the microwell, using photolithographic techniques similar to those used in the generation of a pattern of reaction pads on a planar support. See, Healey, et al, Science 269: 1078-1080 (1995); Munkholm and Walt, Anal. Chem. 58: 1427-1430 (1986), and Bronk, et al., Anal. Chem. 67: 2750-2757 (1995).

The opposing side of the optical fiber wafer (i.e., the non-etched side) is typically highly polished so as to allow optical-coupling (e.g., by immersion oil or other optical coupling fluids) to a second, optical fiber bundle. This second optical fiber bundle exactly matches the diameter of the optical wafer containing the reaction chambers, and serve to act as a conduit for the transmission of light product to the attached detection device, such as a CCD imaging system or camera.

In one preferred embodiment, the fiber optic wafer is thoroughly cleaned, e.g. by serial washes in 15% H₂O₂/15%NH₄OH volume:volume in aqueous solution, then six deionized water rinses, then 0.5M EDTA, then six deionized water, then 15% H₂O₂/15%NH₄OH, then six deionized water (one-half hour incubations in each wash).

The surface of the fiber optic wafer is preferably coated to facilitate its use in the sequencing reactions. A coated surface is preferably optically transparent, allows for easy attachment of proteins and nucleic acids, and does not negatively affect the activity of immobilized proteins. In addition, the surface preferably minimizes non-specific absorption of macromolecules and increases the stability of linked macromolecules (e.g., attached nucleic acids and proteins).

Suitable materials for coating the array include, e.g., plastic (e.g. polystyrene). The plastic can be preferably spin-coated or sputtered (0.1 μm thickness). Other materials for coating the array include gold layers, e.g. 24 karat gold, 0.1 μm thickness, with adsorbed self-assembling monolayers of long chain thiol alkanes. Biotin is then coupled covalently to the surface and saturated with a biotin-binding protein (e.g. streptavidin or avidin).

Coating materials can additionally include those systems used to attach an anchor primer to a substrate. Organosilane reagents, which allow for direct covalent coupling of proteins via amino, sulfhydryl or carboxyl groups, can also be used to coat the array. Additional coating substances include photoreactive linkers, e.g. photobiotin, (Amos et al., “Biomaterial Surface Modification Using Photochemical Coupling Technology,” in Encyclopedic Handbook of Biomaterials and Bioengineering, Part A. Materials, Wise et al. (eds.), New York, Marcel Dekker, pp.895926, 1995).

Additional coating materials include hydrophilic polymer gels (polyacrylamide, polysaccharides), which preferably polymerize directly on the surface or polymer chains covalently attached post polymerization (Hjerten, J. Chromatogr. 347,191 (1985); Novotny, Anal. Chem. 62,2478 (1990), as well as pluronic polymers (triblock copolymers, e.g. PPO-PEO-PPO, also known as F-108), specifically adsorbed to either polystyrene or silanized glass surfaces (Ho et al., Langmuir 14:3889-94, 1998), as well as passively adsorbed layers of biotin-binding proteins. The surface can also be coated with an epoxide which allows the coupling of reagents via an amine linkage.

In addition, any of the above materials can be derivatized with one or more functional groups, commonly known in the art for the immobilization of enzymes and nucleotides, e.g. metal chelating groups (e.g. nitrilo triacetic acid, iminodiacetic acid, pentadentate chelator), which will bind 6xHis-tagged proteins and nucleic acids.

Surface coatings can be used that increase the number of available binding sites for subsequent treatments, e.g. attachment of enzymes (discussed later), beyond the theoretical binding capacity of a 2D surface.

In a preferred embodiment, the individual optical fibers utilized to generate the fused optical fiber bundle/wafer are larger in diameter (i.e., 6 μm to 12 μm) than those utilized in the optical imaging system (i.e., 3 μm). Thus, several of the optical imaging fibers can be utilized to image a single reaction site.

IV. Arrays

In one aspect, the invention involves an array including a planar surface with a plurality of reaction chambers disposed thereon, wherein the reaction chambers have a center to center spacing of between 5 to 200 μm and each chamber has a width in at least one dimension of between 0.3 μm and 100 μm. In some embodiments, the array is a planar surface with a plurality of cavities thereon, where each cavity forms an analyte reaction chamber. In a preferred embodiment, the array is fashioned from a sliced fiber optic bundle (i.e., a bundle of fused fiber optic cables) and the reaction chambers are formed by etching one surface of the fiber optic reactor array (“FORA”). The cavities can also be formed in the substrate via etching, molding or micromachining.

Specifically, each reaction chamber in the array typically has a width in at least one dimension of between 0.3 μm and 100 μm, preferably between 0.3 μm and 20 μm, most preferably between 0.3 μm and 10 μm. In a separate embodiment, we contemplate larger reaction chambers, preferably having a width in at least one dimension of between 20 μm and 70 μm.

The array typically contains more than 1,000 reaction chambers, preferably more than 400,000, more preferably between 400,000 and 20,000,000, and most preferably between 1,000,000 and 16,000,000 cavities or reaction chambers. The shape of each cavity is frequently substantially hexagonal, but the cavities can also be cylindrical. In some embodiments, each cavity has a smooth wall surface, however, we contemplate that each cavity may also have at least one irregular wall surface. The bottom of each of the cavities can be planar or concave.

The array is typically constructed to have cavities or reaction chambers with a center-to-center spacing between 10 to 150 μm, preferably between 50 to 100 μm.

Each cavity or reaction chamber typically has a depth of between 10 μm and 100 μm; alternatively, the depth is between 0.25 and 5 times the size of the width of the cavity, preferably between 0.3 and 1 times the size of the width of the cavity.

In one embodiment, the arrays described herein typically include a planar top surface and a planar bottom surface, which is optically conductive such that optical signals from the reaction chambers can be detected through the bottom planar surface. In these arrays, typically the distance between the top surface and the bottom surface is no greater than 10 cm, preferably no greater than 2 cm, and usually between 0.5 mm to 5 mm.

In one embodiment, each cavity of the array contains reagents for analyzing a nucleic acid or protein. The array can also include a second surface spaced apart from the planar array and in opposing contact therewith such that a flow chamber is formed over the array.

In another aspect, the invention involves an array means for carrying out separate parallel common reactions in an aqueous environment, wherein the array means includes a substrate having at least 1,000 discrete reaction chambers. These chambers contain a starting material that is capable of reacting with a reagent. Each of the reaction chambers are dimensioned such that when one or more fluids containing at least one reagent is delivered into each reaction chamber, the diffusion time for the reagent to diffuse out of the well exceeds the time required for the starting material to react with the reagent to form a product. The reaction chambers can be formed by generating a plurality of cavities on the substrate, or by generating discrete patches on a planar surface, the patches having a different surface chemistry than the surrounding planar surface.

In one embodiment, each cavity or reaction chamber of the array contains reagents for analyzing a nucleic acid or protein. Typically those reaction chambers that contain a nucleic acid (not all reaction chambers in the array are required to) contain only a single species of nucleic acid (i.e., a single sequence that is of interest). There may be a single copy of this species of nucleic acid in any particular reaction chamber, or they may be multiple copies. It is generally preferred that a reaction chamber contain at least 100 copies of the nucleic acid sequence, preferably at least 100,000 copies, and most preferably between 100,000 to 1,000,000 copies of the nucleic acid. The ordinarily skilled artisan will appreciate that changes in the number of copies of a nucleic acid species in any one reaction chamber will affect the number of photons generated in a pyrosequencing reaction, and can be routinely adjusted to provide more or less photon signal as is required. In one embodiment the nucleic acid species is amplified to provide the desired number of copies using PCR, RCA, ligase chain reaction, other isothermal amplification, or other conventional means of nucleic acid amplification. In one embodiment, the nucleic acid is single stranded. In other embodiments the single stranded DNA is a concatamer with each copy covalently liked end to end.

V. Delivery Means

An example of the means for delivering reactants to the reaction chamber is the perfusion chamber of the present invention which includes a sealed compartment with transparent upper and lower slide. It is designed to allow flow of solution over the surface of the substrate surface and to allow for fast exchange of reagents. Thus, it is suitable for carrying out, for example, the sequencing reactions with reversibly labeled nucleotides. The shape and dimensions of the chamber can be adjusted to optimize reagent exchange to include bulk flow exchange, diffusive exchange, or both in either a laminar flow or a turbulent flow regime.

The correct exchange of reactants to the reaction chamber is important for accurate measurements in the present invention. In the absence of convective flow of bulk fluid, transport of reaction participants (and cross-contamination or “cross-talk” between adjacent reaction sites or microvessels) can take place only by diffusion. If the reaction site is considered to be a point source on a 2-D surface, the chemical species of interest (e.g., a reaction product) will diffuse radially from the site of its production, creating a substantially hemispherical concentration field above the surface.

The distance that a chemical entity can diffuse in any given time t may be estimated in a crude manner by considering the mathematics of diffusion (Crank, The Mathematics of Diffusion, 2^{nd} ed. 1975). The rate of diffusive transport in any given direction×(cm) is given by Fick's law as

$$j = - D \frac{\partial C}{\partial x} \qquad Eq . 1$$

where j is the flux per unit area (g-mol/cm²-s) of a species with diffusion coefficient D (cm²/s), and ∂C/∂x is the concentration gradient of that species. The mathematics of diffusion are such that a characteristic or “average” distance an entity can travel by diffusion alone scales with the one-half power of both the diffusion coefficient and the time allowed for diffusion to occur. Indeed, to order of magnitude, this characteristic diffusion distance can be estimated as the square root of the product of the diffusion coefficient and time—as adjusted by a numerical factor of order unity that takes into account the particulars of the system geometry and initial and/or boundary conditions imposed on the diffusion process.

It will be convenient to estimate this characteristic diffusion distance as the root-mean-square distance dᵣₘₛ that a diffusing entity can travel in time t:

dᵣₘₛ=\sqrt{2Dt} Eq. 2

As stated above, the distance that a diffusing chemical typically travels varies with the square root of the time available for it to diffuse—and inversely, the time required for a diffusing chemical to travel a given distance scales with the square of the distance to be traversed by diffusion. Thus, for a simple, low-molecular-weight biomolecule characterized by a diffusion coefficient D of order 1·10⁻⁵ cm²/s, the root-mean-square diffusion distances dᵣₘₛ that can be traversed in time intervals of 0.1 s, 1.0 s, 2.0 s, and 10 s are estimated by means of Equation 2 as 14 μm, 45 μm, 63 μm, and 141 μm, respectively.

The relative importance of convection and diffusion in a transport process that involves both mechanisms occurring simultaneously can be gauged with the aid of a dimensionless number—namely, the Peclet number Pe. This Peclet number can be viewed as a ratio of two rates or velocities—namely, the rate of a convective flow divided by the rate of a diffusive “flow” or flux. More particularly, the Peclet number is a ratio of a characteristic flow velocity V (in cm/s) divided by a characteristic diffusion velocity D/L (also expressed in units of cm/s)—both taken in the same direction:

$$P e = \frac{V L}{D} \qquad Eq . 3$$

In Equation 3, V is the average or characteristic speed of the convective flow, generally determined by dividing the volumetric flow rate Q (in cm³/s) by the cross-sectional area A (cm²) available for flow. The characteristic length L is a representative distance or system dimension measured in a direction parallel to the directions of flow and of diffusion (i.e., in the direction of the steepest concentration gradient) and selected to be representative of the typical or “average” distance over which diffusion occurs in the process. And finally D (cm²/s) is the diffusion coefficient for the diffusing species in question. (An alternative but equivalent formulation of the Peclet number Pe views it as the ratio of two characteristic times—namely, of representative times for diffusion and convection. Equation 3 for the Peclet number can equally well be obtained by dividing the characteristic diffusion time L²/D by the characteristic convection time L/V.)

The convective component of transport can be expected to dominate over the diffusive component in situations where the Peclet number Pe is large compared to unity. Conversely, the diffusive component of transport can be expected to dominate over the convective component in situations where the Peclet number Pe is small compared to unity. In extreme situations where the Peclet number is either very much larger or very much smaller than one, transport may be accurately presumed to occur either by convection or by diffusion alone, respectively. Finally, in situations where the estimated Peclet number is of order unity, then both convection and diffusion can be expected to play significant roles in the overall transport process.

The diffusion coefficient of a typical low-molecular-weight biomolecule will generally be of the order of 10⁻⁵ cm²/s (e.g., 0.52·10⁻⁵ cm/s for sucrose, and 1.06·10⁻⁵ cm/s for glycine). Thus, for reaction centers, cavities, or wells separated by a distance of 100 μm (i.e., 0.01 cm), the Peclet number Pe for low-molecular-weight solutes such as these will exceed unity for flow velocities greater than about 10 μm/sec (0.001 cm/s). For cavities separated by only 10 μm (i.e., 0.001 cm), the Peclet number Pe for low-molecular-weight solutes will exceed unity for flow velocities greater than about 100 μm/sec (0.01 cm/s). Convective transport is thus seen to dominate over diffusive transport for all but very slow flow rates and/or very short diffusion distances.

Where the molecular weight of a diffusible species is substantially larger—for example as it is with large biomolecules like DNA/RNA, DNA fragments, oligonucleotides, proteins, and constructs of the former—then the species diffusivity will be corresponding smaller, and convection will play an even more important role relative to diffusion in a transport process involving both mechanisms. For instance, the aqueous-phase diffusion coefficients of proteins fall in about a 10-fold range (Tanford, Physical Chemistry of Macromolecules, 1961). Protein diffusivities are bracketed by values of 1.19×10⁻⁶ cm²/s for ribonuclease (a small protein with a molecular weight of 13,683 Daltons) and 1.16×10⁻⁷ cm²/s for myosin (a large protein with a molecular weight of 493,000 Daltons). Still larger entities (e.g., tobacco mosaic virus or TMV at 40.6 million Daltons) are characterized by still lower diffusivities (in particular, 4.6×10⁻⁸ cm²/s for TMV) (Lehninger, Biochemistry, 2^{nd} ed. 1975). The fluid velocity at which convection and diffusion contribute roughly equally to transport (i.e., Pe of order unity) scales in direct proportion to species diffusivity.

With the aid of the Peclet number formalism it is possible to gauge the impact of convection on reactant supply to—and product removal from—reaction chambers, cavities or wells. On the one hand, it is clear that even modest convective flows can appreciably increase the speed at which reactants are delivered to the interior of the cavities in an array or FORA. In particular, suppose for the sake of simplicity that the criteria for roughly equal convective and diffusive flows is considered to be Pe=1. One may then estimate that a convective flow velocity of the order of only 0.004 cm/s will suffice to carry reactant into a 25-μm-deep well at roughly the same rate as it could be supplied to the bottom of the well by diffusion alone, given an assumed value for reactant diffusivity of 1×10⁻⁵ cm²/s. The corresponding flow velocity required to match the rate of diffusion of such a species from the bottom to the top of a 2.5-μm-deep microwell is estimated to be of order 0.04 cm/s. Flow velocities through a FORA much higher than this are possible, thereby illustrating the degree to which a modest convective flow can augment the diffusive supply of reactants to FORA reaction centers, cavities or wells.

The perfusion chamber is preferably detached from the imaging system while it is being prepared and only placed on the imaging system when sequencing analysis is performed. In one embodiment, the solid support (i.e., a DNA chip or glass slide) is held in place by a metal or plastic housing, which may be assembled and disassembled to allow replacement of said solid support. The lower side of the solid support of the perfusion chamber carries the reaction chamber array and, with a traditional optical-based focal system, a high numerical aperture objective lens is used to focus the image of the reaction center array onto the CCD imaging system.

An alternative system for the analysis is to use an array format wherein samples are distributed over a surface, for example a microfabricated chip, and thereby an ordered set of samples may be immobilized in a 2-dimensional format. Many samples can thereby be analyzed in parallel. Using the method of the invention, many immobilized templates may be analyzed in this was by allowing the solution containing the enzymes and one nucleotide to flow over the surface and then detecting the signal produced for each sample. This procedure can then be repeated. Alternatively, several different oligonucleotides complementary to the template may be distributed over the surface followed by hybridization of the template. Incorporation of deoxynucleotides or dideoxynucleotides may be monitored for each oligonucleotide by the signal produced using the various oligonucleotides as primer. By combining the signals from different areas of the surface, sequence-based analyses may be performed by four cycles of polymerase reactions using the various dideoxynucleotides.

When the support is in the form of a cavitated array, e.g., in the termini of a FORA or other array of microwells, suitable delivery means for reagents include flowing and washing and also, e.g., flowing, spraying, electrospraying, ink jet delivery, stamping, ultrasonic atomization (Sonotek Corp., Milton, N.Y.) and rolling. Preferably, all reagent solutions contain 10-20% ethylene glycol to minimize evaporation. When spraying is used, reagents are delivered to the FORA surface in a homogeneous thin layer produced by industrial type spraying nozzles (Spraying Systems, Co., Wheaton, Ill.) or atomizers used in thin layer chromatography (TLC), such as CAMAG TLC Sprayer (Camag Scientific Inc., Wilmington, N.C.). These sprayers atomize reagents into aerosol spray particles in the size range of 0.3 to 10 μm.

Electrospray deposition (ESD) of protein and DNA solutions is currently used to generate ions for mass spectrometric analysis of these molecules. Deposition of charged electrospray products on certain areas of a FORA substrate under control of electrostatic forces is suggested. It was also demonstrated that the ES-deposited proteins and DNA retain their ability to specifically bind antibodies and matching DNA probes, respectively, enabling use of the ESD fabricated matrixes in Dot Immuno-Binding (DIB) and in DNA hybridization assays. (Morozov and Morozova Anal. Chem. 71(15):3110-7 (1999)).

Ink jet delivery is applicable to protein solutions and other biomacromolecules, as documented in the literature (e.g. Roda et al., Biotechniques 28(3): 492-6 (2000)). It is also commercially available e.g. from MicroFab Technologies, Inc. (Plano, Tex.).

Reagent solutions can alternatively be delivered to the FORA surface by a method similar to lithography. Rollers (stamps; hydrophilic materials should be used) would be first covered with a reagent layer in reservoirs with dampening sponges and then rolled over (pressed against) the FORA surface.

Successive reagent delivery steps are preferably separated by wash steps using techniques commonly known in the art. These washes can be performed, e.g., using the above described methods, including high-flow sprayers or by a liquid flow over the FORA or microwell array surface. The washes can occur in any time period after the starting material has reacted with the reagent to form a product in each reaction chamber but before the reagent delivered to any one reaction chamber has diffused out of that reaction chamber into any other reaction chamber. In one embodiment, any one reaction chamber is independent of the product formed in any other reaction chamber, but is generated using one or more common reagents.

The invention also provides a method for delivering nucleic acid sequencing enzymes to an array. In some embodiments, one of the nucleic acid sequencing enzymes can be a polypeptide with sulfurylase activity or the nucleic acid sequencing enzyme can be a polypeptide with luciferase activity. In another embodiment, one of the nucleic acid sequencing enzymes can be a polypeptide with both sulfurylase and luciferase activity. In a more preferred embodiment, the reagent can be suitable for use in a nucleic acid sequencing reaction.

In a preferred embodiment, one or more reagents are delivered to an array immobilized or attached to a population of mobile solid supports, e.g., a bead or microsphere. The bead or microsphere need not be spherical, irregular shaped beads may be used. They are typically constructed from numerous substances, e.g., plastic, glass or ceramic and bead sizes ranging from nanometers to millimeters depending on the width of the reaction chamber. Preferably, the diameter of each mobile solid support can be between 0.01 and 0.1 times the width of each cavity. Various bead chemistries can be used e.g., methylstyrene, polystyrene, acrylic polymer, latex, paramagnetic, thoria sol, carbon graphite and titanium dioxide. The construction or chemistry of the bead can be chosen to facilitate the attachment of the desired reagent.

In another embodiment, the bioactive agents are synthesized first, and then covalently attached to the beads. As is appreciated by someone skilled in the art, this will be done depending on the composition of the bioactive agents and the beads. The functionalization of solid support surfaces such as certain polymers with chemically reactive groups such as thiols, amines, carboxyls, etc. is generally known in the art. Accordingly, “blank” beads may be used that have surface chemistries that facilitate the attachment of the desired functionality by the user. Additional examples of these surface chemistries for blank beads include, but are not limited to, amino groups including aliphatic and aromatic amines, carboxylic acids, aldehydes, amides, chloromethyl groups, hydrazide, hydroxyl groups, sulfonates and sulfates.

These functional groups can be used to add any number of different candidate agents to the beads, generally using known chemistries. For example, candidate agents containing carbohydrates may be attached to an amino-functionalized support; the aldehyde of the carbohydrate is made using standard techniques, and then the aldehyde is reacted with an amino group on the surface. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated here by reference) which can be used to attach cysteine containing proteinaceous agents to the support. Alternatively, an amino group on the candidate agent may be used for attachment to an amino group on the surface. For example, a large number of stable bifunctional groups are well known in the art, including homobifunctional and heterobifunctional linkers (see Pierce Catalog and Handbook, pages 155-200). In an additional embodiment, carboxyl groups (either from the surface or from the candidate agent) may be derivatized using well known linkers (see Pierce catalog). For example, carbodiimides activate carboxyl groups for attack by good nucleophiles such as amines (see Torchilin et al., Critical Rev. Therapeutic Drug Carrier Systems, 7(4):275-308 (1991)). Proteinaceous candidate agents may also be attached using other techniques known in the art, for example for the attachment of antibodies to polymers; see Slinkin et al., Bioconj. Chem. 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994). It should be understood that the candidate agents may be attached in a variety of ways, including those listed above. Preferably, the manner of attachment does not significantly alter the functionality of the candidate agent; that is, the candidate agent should be attached in such a flexible manner as to allow its interaction with a target.

Specific techniques for immobilizing enzymes on beads are known in the prior art. In one case, NH₂ surface chemistry beads are used. Surface activation is achieved with a 2.5% glutaraldehyde in phosphate buffered saline (10 mM) providing a pH of 6.9 (138 mM NaCl, 2.7 mM KCl). This mixture is stirred on a stir bed for approximately 2 hours at room temperature. The beads are then rinsed with ultrapure water plus 0.01% Tween 20 (surfactant) −0.02%, and rinsed again with a pH 7.7 PBS plus 0.01% Tween 20. Finally, the enzyme is added to the solution, preferably after being prefiltered using a 0.45 μm amicon micropure filter.

The population of mobile solid supports are disposed in the reaction chambers. In some embodiments, 5% to 20% of the reaction chambers can have a mobile solid support with at least one reagent immobilized thereon, 20% to 60% of the reaction chambers can have a mobile solid support with at least one reagent immobilized thereon or 50% to 100% of the reaction chambers can have a mobile solid support with at least one reagent immobilized thereon. Preferably, at least one reaction chamber has a mobile solid support having at least one reagent immobilized thereon and the reagent is suitable for use in a nucleic acid sequencing reaction.

The invention also provides a method for detecting or quantifying labels activity using a mobile solid support; preferably the label can be detected or quantified as part of a nucleic acid sequencing reaction.

The solid support (FOR A) may be optically linked to an imaging system which includes a CCD system in association with conventional optics or a fiber optic bundle. In one embodiment the perfusion chamber substrate includes a fiber optic array wafer such that light generated near the aqueous interface is transmitted directly through the optical fibers to the exterior of the substrate or chamber. When the CCD system includes a fiber optic connector, imaging can be accomplished by placing the perfusion chamber substrate in direct contact with the connector. Alternatively, conventional optics can be used to image the light, e.g., by using a 1-1 magnification high numerical aperture lens system, from the exterior of the fiber optic substrate directly onto the CCD sensor. When the substrate does not provide for fiber optic coupling, a lens system can also be used as described above, in which case either the substrate or the perfusion chamber cover is optically transparent. An exemplary CCD imaging system is described above.

The imaging system is used to collect light from the reactors on the substrate surface. Light can be imaged, for example, onto a CCD using a high sensitivity low noise apparatus known in the art. For fiber-optic based imaging, it is preferable to incorporate the optical fibers directly into the cover slip or for a FORA to have the optical fibers that form the microwells also be the optical fibers that convey light to the detector.

The imaging system is linked to a computer control and data collection system. In general, any commonly available hardware and software package can be used. The computer control and data collection system is also linked to the conduit 200 to control reagent delivery.

The photons generated directly or indirectly by the reversible label are captured by the CCD only if they pass through a focusing device (e.g., an optical lens or optical fiber) and are focused upon a CCD element. However, the emitted photons will escape equally in all directions. In order to maximize their subsequent “capture” and quantitation when utilizing a planar array (e.g., a DNA chip), it is preferable to collect the photons as close as possible to the point at which they are generated, e.g. immediately at the planar solid support. This is accomplished by either: (i) utilizing optical immersion oil between the cover slip and a traditional optical lens or optical fiber bundle or, preferably, (ii) incorporating optical fibers directly into the cover slip itself. Similarly, when a thin, optically transparent planar surface is used, the optical fiber bundle can also be placed against its back surface, eliminating the need to “image” through the depth of the entire reaction/perfusion chamber.

VI. Detection Means

The reaction event, e.g., photons generated by luciferase, may be detected and quantified using a variety of detection apparatuses, e.g., a photomultiplier tube, a CCD, CMOS, absorbance photometer, a luminometer, charge injection device (CID), or other solid state detector, as well as the apparatuses described herein. In a preferred embodiment, the quantitation of the emitted photons is accomplished by the use of a CCD camera fitted with a fused fiber optic bundle. In another preferred embodiment, the quantitation of the emitted photons is accomplished by the use of a CCD camera fitted with a microchannel plate intensifier. A back-thinned CCD can be used to increase sensitivity. CCD detectors are described in, e.g., Bronks, et al., 1995. Anal. Chem. 65: 2750-2757.

An exemplary CCD system is a Spectral Instruments, Inc. (Tucson, Ariz.) Series 600 4-port camera with a Lockheed-Martin LM485 CCD chip and a 1-1 fiber optic connector (bundle) with 6-8 μm individual fiber diameters. This system has 4096×4096, or greater than 16 million pixels and has a quantum efficiency ranging from 10% to >40%. Thus, depending on wavelength, as much as 40% of the photons imaged onto the CCD sensor are converted to detectable electrons.

In other embodiments, a fluorescent moiety can be used as a label and the detection of a reaction event can be carried out using a confocal scanning microscope to scan the surface of an array with a laser or other techniques such as scanning near-field optical microscopy (SNOM) are available which are capable of smaller optical resolution, thereby allowing the use of “more dense” arrays. For example, using SNOM, individual polynucleotides may be distinguished when separated by a distance of less than 100 nm, e.g., 10 nm×10 nm. Additionally, scanning tunneling microscopy (Binning et al., Helvetica Physica Acta, 55:726-735, 1982) and atomic force microscopy (Hanswa et al., Annu Rev Biophys Biomol Struct, 23:115-139, 1994) can be used.

The invention provides an apparatus for simultaneously monitoring an array of reaction chambers for light indicating that a reaction is taking place at a particular site. The apparatus can include an array of reaction chambers formed from a planar substrate having a plurality of cavitated surfaces, each cavitated surface forming a reaction chamber adapted to contain analytes. The reaction chambers can have a center-to-center spacing of between 5 to 200 μm and the array can have more than 400,000 discrete reaction chambers. The apparatus can also include an optically sensitive device arranged so that in use the light from a particular reaction chamber will impinge upon a particular predetermined region of said optically sensitive device. The apparatus can further include a means for determining the light level impinging upon each predetermined region and a means to record the variation of said light level with time for each of said reaction chamber.

The invention also provides an analytic sensor, which can include an array formed from a first bundle of optical fibers with a plurality of cavitated surfaces at one end thereof, each cavitated surface forming a reaction chamber adapted to contain analytes. The reaction chambers can have a center-to-center spacing of between 5 to 200 μm and the array can have more than 400,000 discrete reaction chambers. The analytic sensor can also include an enzymatic or fluorescent means for generating light in the reaction chambers. The analytic sensor can further include a light detection means comprising a light capture means and a second fiber optic bundle for transmitting light to the light detecting means. The second fiber optic bundle can be in optical contact with the array, such that light generated in an individual reaction chamber is captured by a separate fiber or groups of separate fibers of the second fiber optic bundle for transmission to the light capture means. The light capture means can be a CCD camera as described herein. The reaction chambers can contain one or more mobile solid supports with a bioactive agent immobilized thereon. In some embodiments, the analytic sensor is suitable for use in a biochemical assay or suitable for use in a cell-based assay.

VII. Methods of Sequencing Nucleic Acids

The invention also provides a method for sequencing nucleic acids which generally comprises (a) providing one or more nucleic acid anchor primers and a plurality of single-stranded circular nucleic acid templates disposed within a plurality of reaction chambers or cavities; (b) annealing an effective amount of the nucleic acid anchor primer to at least one of the single-stranded circular templates to yield a primed anchor primer-circular template complex; (c) combining the primed anchor primer-circular template complex with a polymerase to form an extended anchor primer covalently linked to multiple copies of a nucleic acid complementary to the circular nucleic acid template; (d) annealing an effective amount of a sequencing primer to one or more copies of said covalently linked complementary nucleic acid; (e) extending the sequencing primer with a polymerase and a predetermined nucleotide triphosphate which is reversibly labeled and optionally reversibly terminated to yield a sequencing product and (f) detecting the label thereby determining the sequence of the nucleic acid. Since the label is reversible, the incorporated nucleotide may be unlabeled. That is, if desired, the label may be removed and/or inactivated and step (e) and (f) may be repeated. If the nucleotide is reversibly terminated, the termination may be reversed and step (e) and (f) may be repeated. It is understood that some sequencing projects, such as the detection of SNPs, the determination of one base of DNA is sufficient. In those cases, there may not be a need to repeat steps (e) and (f). In this example, termination of a nucleotide refers to an alteration of the molecular structure of a nucleotide such that after its incorporation into a nucleic acid strand no other nucleotide triphosphate may be added after the nucleotide. A common terminated nucleotide (nucleotide triphosphate) is dideoxy nucleotide triphosphates. A reversibly terminated nucleotide would thus act like a dideoxy nucleotide in that it will be incorporated into a nucleic acid strand but after its incorporation, no additional nucleotide may be added enzymatically after (3′) to the dideoxy nucleotide. However, since the termination is reversible, it can be removed to allow chain elongation of the nucleic acid strand pass the reversibly terminated nucleotide. The methods of the invention, discussed throughout this patent, can be carried out in separate parallel common reactions in an aqueous environment.

In another aspect, the invention includes a method of determining the base sequence of a plurality of nucleotides on an array, which generally comprises (a) providing a plurality of sample DNAs, each disposed within a plurality of cavities on a planar surface; (b) adding a reversibly labeled and optionally reversibly terminated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the a reversibly labeled and optionally reversibly terminated nucleoside 5′-triphosphate precursor onto the 3′-end of the primer strands, provided the nitrogenous base of the nucleoside 5′-triphosphate precursor is complementary to the nitrogenous base of the unpaired nucleotide residue of the templates; (c) detecting the label and thereby detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; and (d) sequentially repeating steps (b) and (c), wherein each sequential repetition adds and, detects the incorporation of one type of a reversibly labeled and optionally reversibly terminated nucleoside 5′-triphosphate precursor of known nitrogenous base composition; and (e) determining the base sequence of the unpaired nucleotide residues of the template in each reaction chamber from the sequence of incorporation of said nucleoside precursors.

In one embodiment of the invention, the anchor primer is linked to a particle. The anchor primer could be linked to the particle prior to or after formation of the extended anchor primer. The sequencing reaction using a reversibly labeled and optionally reversibly terminated nucleotide will incorporate a label into the elongated nucleic acid. This label is used to generate light for detection.

In another aspect, the invention involves, a method of determining the base sequence of a plurality of nucleotides on an array. The method includes providing a plurality of sample DNAs, each disposed within a plurality of cavities on a planar surface, each cavity forming an analyte reaction chamber, wherein the reaction chambers have a center to center spacing of between 5 to 200 μm. Then an a reversibly labeled and optionally reversibly terminated nucleotide 5′-triphosphate precursor of one known nitrogenous base is added to a reaction mixture in each reaction chamber. Each reaction mixture includes a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the a reversibly labeled and optionally reversibly terminated nucleoside 5′-triphosphate precursor onto the 3′-end of the primer strands, provided the nitrogenous base of the nucleoside 5′-triphosphate precursor is complementary to the nitrogenous base of the unpaired nucleotide residue of the templates. Then it is detected whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor. Then these steps are sequentially repeated, wherein each sequential repetition adds and, detects the incorporation of one type of a reversibly labeled and optionally reversibly terminated nucleoside 5′-triphosphate precursor of known nitrogenous base composition. The base sequence of the unpaired nucleotide residues of the template in each reaction chamber is then determined from the sequence of incorporation of the nucleoside precursors.

In another aspect, the invention involves a method for determining the nucleic acid sequence in a template nucleic acid polymer. The method includes introducing a plurality of template nucleic acid polymers into a plurality of cavities on a planar surface, each cavity forming an analyte reaction chamber, wherein the reaction chambers have a center to center spacing of between 5 to 200 μm. Each reaction chamber also has a polymerization environment in which the nucleic acid polymer will act as a template polymer for the synthesis of a complementary nucleic acid polymer when nucleotides are added. A series of feedstocks is successively provided to the polymerization environment, each feedstock having a reversibly labeled and optionally reversibly terminated nucleotide selected from among the nucleotides from which the complementary nucleic acid polymer will be formed, such that if the nucleotide in the feedstock is complementary to the next nucleotide in the template polymer to be sequenced the nucleotide will be incorporated into the complementary polymer and the reversible label will also be incorporated. Then the label is detected to determine the identity of each nucleotide in the complementary polymer and thus the sequence of the template polymer.

In another aspect, the invention involves, a method of identifying the base in a target position in a DNA sequence of sample DNA. The method includes providing a sample of DNA disposed within a plurality of cavities on a planar surface, each cavity forming an analyte reaction chamber, wherein the reaction chambers have a center to center spacing of between 5 to 200 μm, the DNA being rendered single stranded either before or after being disposed in the reaction chambers. An extension primer is then provided which hybridizes to the immobilized single-stranded DNA at a position immediately adjacent to the target position. The immobilized single-stranded DNA is subjected to a polymerase reaction in the presence of a predetermined nucleotide triphosphate, wherein if the predetermined nucleotide triphosphate is incorporated onto the 3′ end of the sequencing primer then a sequencing reaction byproduct is formed. The sequencing reaction byproduct is then identified, thereby determining the nucleotide complementary to the base at the target position.

In another aspect, the invention involves a method of identifying a base at a target position in a sample DNA sequence. The method includes providing sample DNA disposed within a plurality of cavities on a planar surface, each cavity forming an analyte reaction chamber, wherein the reaction chambers have a center to center spacing of between 5 to 200 μm, the DNA being rendered single stranded either before or after being disposed in the reaction chambers and providing an extension primer which hybridizes to the sample DNA immediately adjacent to the target position. The sample DNA sequence and the extension primer are then subjected to a polymerase reaction in the presence of a a reversibly labeled and optionally reversibly terminated nucleotide triphosphate whereby the nucleotide triphosphate (and hence the label) will only become incorporated if it is complementary to the base in the target position, the nucleotide triphosphate being added either to separate aliquots of sample-primer mixture or successively to the same sample-primer mixture. The incorporated label is then detected to indicate which nucleotide is incorporated.

In another aspect, the invention involves a method of identifying a base at a target position in a single-stranded sample DNA sequence. The method includes providing an extension primer which hybridizes to sample DNA immediately adjacent to the target position, the sample DNA disposed within a plurality of cavities on a planar surface, each cavity forming an analyte reaction chamber, wherein the reaction chambers have a center to center spacing of between 5 to 200 μm, the DNA being rendered single stranded either before or after being disposed in the reaction chambers. The sample DNA and extension primer is subjected to a polymerase reaction in the presence of a predetermined deoxynucleotide or dideoxynucleotide which is reversibly labeled. The label will only become incorporated if it is complementary to the base in the target position, the predetermined deoxynucleotides or dideoxynucleotides being added either to separate aliquots of sample-primer mixture or successively to the same sample-primer mixture. Any incorporated label is detected enzymatically to indicate which deoxynucleotide or dideoxynucleotide is incorporated.

In another aspect, the invention involves a method for sequencing a nucleic acid. The method includes providing one or more nucleic acid anchor primers; and a plurality of single-stranded circular nucleic acid templates disposed within a plurality of cavities on a planar surface, each cavity forming an analyte reaction chamber, wherein the reaction chambers have a center to center spacing of between 5 to 200 μm. An effective amount of the nucleic acid anchor primer is annealed to at least one of the single-stranded circular templates to yield a primed anchor primer-circular template complex. The primed anchor primer-circular template complex is then combined with a polymerase to form an extended anchor primer covalently linked to multiple copies of a nucleic acid complementary to the circular nucleic acid template; followed by annealing of an effective amount of a sequencing primer to one or more copies of the covalently linked complementary nucleic acid. The sequencing primer is then extended with a polymerase and a predetermined reversibly labeled and optionally reversibly terminated nucleotide triphosphate to yield a sequencing product. If the predetermined nucleotide triphosphate is incorporated onto the 3′ end of the sequencing primer, the label will become a part of the primer.. Then the label is detected and the sequence of the nucleic acid may be determined.

VIII. Structure of Anchor Primers

The anchor primers of the invention generally comprise a stalk region and at least one adaptor region. In a preferred embodiment the anchor primer contains at least two contiguous adapter regions. The stalk region is present at the 5′ end of the anchor primer and includes a region of nucleotides for attaching the anchor primer to the solid substrate.

The adaptor region(s) comprise nucleotide sequences that hybridize to a complementary sequence present in one or more members of a population of nucleic acid sequences. In some embodiments, the anchor primer includes two adjoining adaptor regions, which hybridize to complementary regions ligated to separate ends of a target nucleic acid sequence. In additional embodiments, the adapter regions in the anchor primers are complementary to non-contiguous regions of sequence present in a second nucleic acid sequence. Each adapter region, for example, can be homologous to each terminus of a fragment produced by digestion with one or more restriction endonucleases. The fragment can include, e.g., a sequence known or suspected to contain a sequence polymorphism. Additionally, the anchor primer may contain two adapter regions that are homologous to a gapped region of a target nucleic acid sequence, i.e., one that is non-contiguous because of a deletion of one or more nucleotides. When adapter regions having these sequences are used, an aligning oligonucleotide corresponding to the gapped sequence may be annealed to the anchor primer along with a population of template nucleic acid molecules.

The anchor primer may optionally contain additional elements such as one or more restriction enzyme recognition sites, RNA polymerase binding sites, e.g., a T7 promoter site, or sequences present in identified DNA sequences, e.g., sequences present in known genes. The adapter region(s) may also include sequences known to flank sequence polymorphisms. Sequence polymorphisms include nucleotide substitutions, insertions, deletions, or other rearrangements which result in a sequence difference between two otherwise identical nucleic acid sequences. An example of a sequence polymorphism is a single nucleotide polymorphism (SNP).

In general, any nucleic acid capable of base-pairing can be used as an anchor primer. In some embodiments, the anchor primer is an oligonucleotide. As utilized herein the term oligonucleotide includes linear oligomers of natural or modified monomers or linkages, e.g., deoxyribonucleosides, ribonucleosides, anomeric forms thereof, peptide nucleic acids (PNAs), and the like, that are capable of specifically binding to a target polynucleotide by way of a regular pattern of monomer-to-monomer interactions. These types of interactions can include, e.g., Watson-Crick type of base-pairing, base stacking, Hoogsteen or reverse-Hoogsteen types of base-pairing, or the like. Generally, the monomers are linked by phosphodiester bonds, or analogs thereof, to form oligonucleotides ranging in size from, e.g., 3-200, 8-150, 10-100, 20-80, or 25-50 monomeric units. Whenever an oligonucleotide is represented by a sequence of letters, it is understood that the nucleotides are oriented in the 5′→3′ direction, from left-to-right, and that the letter “A” donates deoxyadenosine, the letter “T” denotes thymidine, the letter “C” denotes deoxycytosine, and the letter “G” denotes deoxyguanosine, unless otherwise noted herein. The oligonucleotides of the present invention can include non-natural nucleotide analogs. However, where, for example, processing by enzymes is required, or the like, oligonucleotides comprising naturally occurring nucleotides are generally required for maintenance of biological function.

IX. Linking Primers to Solid Substrates

Anchor primers are linked to the solid substrate at the sensitized sites. A region of a solid substrate containing a linked primer is referred to herein as an anchor pad. Thus, by specifying the sensitized states on the solid support, it is possible to form an array or matrix of anchor pads. The anchor pads can be, e.g., small diameter spots etched at evenly spaced intervals on the solid support. The anchor pads can be located at the bottoms of the cavitations or wells if the substrate has been cavitated, etched, or otherwise micromachined as discussed above.

In one embodiment, the anchor primer is linked to a particle. The anchor primer can be linked to the particle prior to formation of the extended anchor primer or after formation of the extended anchor primer.

The anchor primer can be attached to the solid support via a covalent or non-covalent interaction. In general, any linkage recognized in the art can be used. Examples of such linkages common in the art include any suitable metal (e.g., Co²⁺, Ni²⁺)-hexahistidine complex, a biotin binding protein, e.g., NEUTRAVIDIN™ modified avidin (Pierce Chemicals, Rockford, Ill), streptavidin/biotin, avidin/biotin, glutathione S-transferase (GST)/glutathione, monoclonal antibody/antigen, and maltose binding protein/maltose, and pluronic coupling technologies. Samples containing the appropriate tag are incubated with the sensitized substrate so that zero, one, or multiple molecules attach at each sensitized site.

One biotin-(strept-)avidin-based anchoring method uses a thin layer of a photoactivatable biotin analog dried onto a solid surface. (Hengsakul and Cass, 1996. Bioconjugate Chem. 7: 249-254). The biotin analog is then exposed to white light through a mask, so as to create defined areas of activated biotin. Avidin (or streptavidin) is then added and allowed to bind to the activated biotin. The avidin possesses free biotin binding sites which can be utilized to “anchor” the biotinylated oligonucleotides through a biotin-(strept-)avidin linkage.

Alternatively, the anchor primer can be attached to the solid support with a biotin derivative possessing a photo-removable protecting group. This moiety is covalently bound to bovine serum albumin (BSA), which is attached to the solid support, e.g., a glass surface. See Pirrung and Huang, 1996. Bioconjugate Chem. 7: 317-321. A mask is then used to create activated biotin within the defined irradiated areas. Avidin may then be localized to the irradiated area, with biotinylated DNA subsequently attached through a BSA-biotin-avidin-biotin link. If desired, an intermediate layer of silane is deposited in a self-assembled monolayer on a silicon dioxide silane surface that can be patterned to localize BSA binding in defined regions. See e.g., Mooney, et al., 1996. Proc. Natl. Acad. Sci. USA 93: 12287-12291.

In pluronic based attachment, the anchor primers are first attached to the termini of a polyethylene oxide-polypropylene oxide-polyethylene oxide triblock copolymer, which is also known as a pluronic compound. The pluronic moiety can be used to attach the anchor primers to a solid substrate. Pluronics attach to hydrophobic surfaces by virtue of the reaction between the hydrophobic surface and the polypropylene oxide. The remaining polyethylene oxide groups extend off the surface, thereby creating a hydrophilic environment. Nitrilotriacetic acid (NTA) can be conjugated to the terminal ends of the polyethylene oxide chains to allow for hexahistidine tagged anchor primers to be attached. In another embodiment, pyridyl disulfide (PDS) can be conjugated to the ends of the polyethylene chains allowing for attachment of a thiolated anchor primer via a disulfide bond. In one preferred embodiment, Pluronic F108 (BASF Corp.) is used for the attachment.

Each sensitized site on a solid support is potentially capable of attaching multiple anchor primers. Thus, each anchor pad may include one or more anchor primers. It is preferable to maximize the number of pads that have only a single productive reaction center (e.g., the number of pads that, after the extension reaction, have only a single sequence extended from the anchor primer). This can be accomplished by techniques which include, but are not limited to: (i) varying the dilution of biotinylated anchor primers that are washed over the surface; (ii) varying the incubation time that the biotinylated primers are in contact with the avidin surface; (iii) varying the concentration of open- or closed-circular template so that, on average, only one primer on each pad is extended to generate the sequencing template; or (iv) reducing the size of the anchor pad to approach single-molecule dimensions (<1 μm) such that binding of one anchor inhibits or blocks the binding of another anchor (e.g. by photoactivation of a small spot); or (v) reducing the size of the anchor pad such that binding of one circular template inhibits or blocks the binding of a second circular template.

In some embodiments, each individual pad contains just one linked anchor primer. Pads having only one anchor primer can be made by performing limiting dilutions of a selected anchor primer on to the solid support such that, on average, only one anchor primer is deposited on each pad. The concentration of anchor primer to be applied to a pad can be calculated utilizing, for example, a Poisson distribution model.

In order to maximize the number of reaction pads that contain a single anchor primer, a series of dilution experiments are performed in which a range of anchor primer concentrations or circular template concentrations are varied. For highly dilute concentrations of primers, primers and circular templates binding to the same pad will be independent of each other, and a Poisson distribution will characterize the number of anchor primers extended on any one pad. Although there will be variability in the number of primers that are actually extended, a maximum of 37% of the pads will have a single extended anchor primer (the number of pads with a single anchor oligonucleotide). This number can be obtained as follows.

Let Nₚ be the average number of anchor primers on a pad and f be the probability that an anchor primer is extended with a circular template. Then the average number of extended anchor primers per pad is Nₚf, which is defined as the quantity a. There will be variability in the number of primers that are actually extended. In the low-concentration limit, primers and circular templates binding to the same pad will be independent of each other, and a Poisson distribution P(n) will characterize the number of anchor primers n extended on any pad. This distribution may be mathematically defined by: P(n)=(aⁿ/n!)exp(−a), with P(1)=a exp(−a). The probability P(1) assumes its maximum value exp(−1) for a=1, with 37% of pads having a single extended anchor primer.

A range of anchor primer concentrations and circular template concentrations may be subsequently scanned to find a value of Nₚf closest to 1. A preferable method to optimize this distribution is to allow multiple anchor primers on each reaction pad, but use a limiting dilution of circular template so that, on average, only one primer on each pad is extended to generate the sequencing template.

Alternatively, at low concentrations of anchor primers, at most one anchor primer will likely be bound on each reaction pad. A high concentration of circular template may be used so that each primer is likely to be extended.

Where the reaction pads are arrayed on a planar surface or a fiber optic array, the individual pads are approximately 10 μm on a side, with a 100 μm spacing between adjacent pads. Hence, on a 1 cm surface a total of approximately 10,000 microreactors could be deposited, and, according to the Poisson distribution, approximately 3700 of these will contain a single anchor primer. In certain embodiments, after the primer oligonucleotide has been attached to the solid support, modified, e.g., biotinylated, enzymes are deposited to bind to the remaining, unused avidin binding sites on the surface.

In other embodiments multiple anchor primers are attached to any one individual pad in an array. Limiting dilutions of a plurality of circular nucleic acid templates (described in more detail below) may be hybridized to the anchor primers so immobilized such that, on average, only one primer on each pad is hybridized to a nucleic acid template. Library concentrations to be used may be calculated utilizing, for example, limiting dilutions and a Poisson distribution model.

X. Nucleic Acid Templates

The nucleic acid templates that can be sequenced according to the invention, e.g., a nucleic acid library, in general can include open circular or closed circular nucleic acid molecules. A “closed circle” is a covalently closed circular nucleic acid molecule, e.g., a circular DNA or RNA molecule. An “open circle” is a linear single-stranded nucleic acid molecule having a 5′ phosphate group and a 3′ hydroxyl group. In one embodiment, the single stranded nucleic acid contains at least 100 copies of nucleic acid sequence, each copy covalently linked end to end. In some embodiments, the open circle is formed in situ from a linear double-stranded nucleic acid molecule. The ends of a given open circle nucleic acid molecule can be ligated by DNA ligase. Sequences at the 5′ and 3′ ends of the open circle molecule are complementary to two regions of adjacent nucleotides in a second nucleic acid molecule, e.g., an adapter region of an anchor primer, or to two regions that are nearly adjoining in a second DNA molecule. Thus, the ends of the open-circle molecule can be ligated using DNA ligase, or extended by DNA polymerase in a gap-filling reaction. Open circles are described in detail in Lizardi, U.S. Pat. No. 5,854,033. An open circle can be converted to a closed circle in the presence of a DNA ligase (for DNA) or RNA ligase following, e.g., annealing of the open circle to an anchor primer.

If desired, nucleic acid templates can be provided as padlock probes. Padlock probes are linear oligonucleotides that include target-complementary sequences located at each end, and which are separated by a linker sequence. The linkers can be ligated to ends of members of a library of nucleic acid sequences that have been, e.g., physically sheared or digested with restriction endonucleases. Upon hybridization to a target-sequence, the 5′- and 3′-terminal regions of these linear oligonucleotides are brought in juxtaposition. This juxtaposition allows the two probe segments (if properly hybridized) to be covalently-bound by enzymatic ligation (e.g., with T4 DNA ligase), thus converting the probes to circularly-closed molecules which are catenated to the specific target sequences (see e.g., Nilsson, et al., 1994. Science 265: 2085-2088). The resulting probes are suitable for the simultaneous analysis of many gene sequences both due to their specificity and selectivity for gene sequence variants (see e.g., Lizardi, et al., 1998. Nat. Genet. 19: 225-232; Nilsson, et al., 1997. Nat. Genet. 16: 252-255) and due to the fact that the resulting reaction products remain localized to the specific target sequences. Moreover, intramolecular ligation of many different probes is expected to be less susceptible to non-specific cross-reactivity than multiplex PCR-based methodologies where non-cognate pairs of primers can give rise to irrelevant amplification products (see e.g., Landegren and Nilsson, 1997. Ann. Med. 29: 585-590).

A starting library can be constructed comprising either single-stranded or double-stranded nucleic acid molecules, provided that the nucleic acid sequence includes a region that, if present in the library, is available for annealing, or can be made available for annealing, to an anchor primer sequence. For example, when used as a template for rolling circle amplification, a region of a double-stranded template needs to be at least transiently single-stranded in order to act as a template for extension of the anchor primer.

Library templates can include multiple elements, including, but not limited to, one or more regions that are complementary to the anchor primer. For example, the template libraries may include a region complementary to a sequencing primer, a control nucleotide region, and an insert sequence comprised of the sequencing template to be subsequently characterized. As is explained in more detail below, the control nucleotide region is used to calibrate the relationship between the amount of byproduct and the number of nucleotides incorporated. As utilized herein the term “complement” refers to nucleotide sequences that are able to hybridize to a specific nucleotide sequence to form a matched duplex.

In one embodiment, a library template includes: (i) two distinct regions that are complementary to the anchor primer, (ii) one region homologous to the sequencing primer, (iii) one optional control nucleotide region, (iv) an insert sequence of, e.g., 30-500, 50-200, or 60-100 nucleotides, that is to be sequenced. The template can, of course, include two, three, or all four of these features.

The template nucleic acid can be constructed from any source of nucleic acid, e.g., any cell, tissue, or organism, and can be generated by any art-recognized method. Suitable methods include, e.g., sonication of genomic DNA and digestion with one or more restriction endonucleases (RE) to generate fragments of a desired range of lengths from an initial population of nucleic acid molecules. Preferably, one or more of the restriction enzymes have distinct four-base recognition sequences. Examples of such enzymes include, e.g., Sau3A1, MspI, and TaqI. Preferably, the enzymes are used in conjunction with anchor primers having regions containing recognition sequences for the corresponding restriction enzymes. In some embodiments, one or both of the adapter regions of the anchor primers contain additional sequences adjoining known restriction enzyme recognition sequences, thereby allowing for capture or annealing to the anchor primer of specific restriction fragments of interest to the anchor primer. In other embodiments, the restriction enzyme is used with a type IIS restriction enzyme.

Alternatively, template libraries can be made by generating a complementary DNA (cDNA) library from RNA, e.g., messenger RNA (mRNA). The cDNA library can, if desired, be further processed with restriction endonucleases to obtain a 3′ end characteristic of a specific RNA, internal fragments, or fragments including the 3′ end of the isolated RNA. Adapter regions in the anchor primer may be complementary to a sequence of interest that is thought to occur in the template library, e.g., a known or suspected sequence polymorphism within a fragment generated by endonuclease digestion.

In one embodiment, an indexing oligonucleotide can be attached to members of a template library to allow for subsequent correlation of a template nucleic acid with a population of nucleic acids from which the template nucleic acid is derived. For example, one or more samples of a starting DNA population can be fragmented separately using any of the previously disclosed methods (e.g., restriction digestion, sonication). An indexing oligonucleotide sequence specific for each sample is attached to, e.g., ligated to, the termini of members of the fragmented population. The indexing oligonucleotide can act as a region for circularization, amplification and, optionally, sequencing, which permits it to be used to index, or code, a nucleic acid so as to identify the starting sample from which it is derived.

Distinct template libraries made with a plurality of distinguishable indexing primers can be mixed together for subsequent reactions. Determining the sequence of the member of the library allows for the identification of a sequence corresponding to the indexing oligonucleotide. Based on this information, the origin of any given fragment can be inferred.

XI. Annealing and Amplification of Primer-Template Nucleic Acid Complexes

Libraries of nucleic acids are annealed to anchor primer sequences using recognized techniques (see, e.g., Hatch, et al., 1999. Genet. Anal. Biomol. Engineer. 15: 35-40; Kool, U.S. Pat. No. 5,714,320 and Lizardi, U.S. Pat. No. 5,854,033). In general, any procedure for annealing the anchor primers to the template nucleic acid sequences is suitable as long as it results in formation of specific, i.e., perfect or nearly perfect, complementarity between the adapter region or regions in the anchor primer sequence and a sequence present in the template library.

A number of in vitro nucleic acid amplification techniques may be utilized to extend the anchor primer sequence. The size of the amplified DNA preferably is smaller than the size of the anchor pad and also smaller than the distance between anchor pads.

The amplification is typically performed in the presence of a polymerase, e.g., a DNA or RNA-directed DNA polymerase, and one, two, three, or four types of nucleotide triphosphates, and, optionally, auxiliary binding proteins. In general, any polymerase capable of extending a primed 3′-OH group can be used a long as it lacks a 3′ to 5′ exonuclease activity. Suitable polymerases include, e.g., the DNA polymerases from Bacillus stearothermophilus, Thermus acquaticus, Pyrococcus furiosis, Thermococcus litoralis, and Thermus thermophilus, bacteriophage T4 and T7, and the E. coli DNA polymerase I Klenow fragment. Suitable RNA-directed DNA polymerases include, e.g., the reverse transcriptase from the Avian Myeloblastosis Virus, the reverse transcriptase from the Moloney Murine Leukemia Virus, and the reverse transcriptase from the Human Immunodeficiency Virus-I.

A number of in vitro nucleic acid amplification techniques have been described. These amplification methodologies may be differentiated into those methods: (i) which require temperature cycling-polymerase chain reaction (PCR) (see e.g., Saiki, et al, 1995. Science 230: 1350-1354), ligase chain reaction (see e.g., Barany, 1991. Proc. Natl. Acad. Sci. USA 88: 189-193; Barringer, et al., 1990. Gene 89: 117-122) and transcription-based amplification (see e.g., Kwoh, et al., 1989. Proc. Natl. Acad. Sci. USA 86: 1173-1177) and (ii) isothermal amplification systems-self-sustaining, sequence replication (see e.g., Guatelli, et al., 1990. Proc. Natl. Acad. Sci. USA 87: 1874-1878); the Qβ replicase system (see e.g., Lizardi, et al., 1988. BioTechnology 6: 1197-1202); strand displacement amplification Nucleic Acids Res. Apr. 11, 1992;20(7):1691-6.; and the methods described in PNAS Jan. 1, 1992;89(1):392-6; and NASBA J Virol Methods. December 1991;35(3):273-86.

Isothermal amplification also includes rolling circle-based amplification (RCA). RCA is discussed in, e.g., Kool, U.S. Pat. No. 5,714,320 and Lizardi, U.S. Pat. No. 5,854,033; Hatch, et al., 1999. Genet. Anal. Biomol. Engineer. 15: 35-40. The result of the RCA is a single DNA strand extended from the 3′ terminus of the anchor primer (and thus is linked to the solid support matrix) and including a concatamer containing multiple copies of the circular template annealed to a primer sequence. Typically, 1,000 to 10,000 or more copies of circular templates, each having a size of, e.g., approximately 30-500, 50-200, or 60-100 nucleotides size range, can be obtained with RCA.

The product of RCA amplification is made as follows. A circular template nucleic acid is annealed to an anchor primer, which has been linked to a surface at its 5′ end and has a free 3′ OH available for extension. The circular template nucleic acid includes two adapter regions which are complementary to regions of sequence in the anchor primer. Also included in the circular template nucleic acid is an insert and a region homologous to a sequencing primer, which is used in the sequencing reactions described below.

Upon annealing, the free 3′-OH on the anchor primer can be extended using sequences within the template nucleic acid. The anchor primer can be extended along the template multiple times, with each iteration adding to the sequence extended from the anchor primer a sequence complementary to the circular template nucleic acid. Multiple iterations, or rounds of amplification may be used to produce the template of the invention. See, U.S. Pat. No. 6,274,320 for a more detailed description.

Circular oligonucleotides that are generated during polymerase-mediated DNA replication are dependent upon the relationship between the template and the site of replication initiation. In double-stranded DNA templates, the critical features include whether the template is linear or circular in nature, and whether the site of initiation of replication (i.e., the replication “fork”) is engaged in synthesizing both strands of DNA or only one. In conventional double-stranded DNA replication, the replication fork is treated as the site at which the new strands of DNA are synthesized. However, in linear molecules (whether replicated unidirectionally or bidirectionally), the movement of the replication fork(s) generate a specific type of structural motif. If the template is circular, one possible spatial orientation of the replicating molecule takes the form of a θ structure.

Alternatively, RCA can occur when the replication of the duplex molecule begins at the origin. Subsequently, a nick opens one of the strands, and the free 3′-terminal hydroxyl moiety generated by the nick is extended by the action of DNA polymerase. The newly synthesized strand eventually displaces the original parental DNA strand. This aforementioned type of replication is known as rolling-circle replication (RCR) because the point of replication may be envisaged as “rolling around” the circular template strand and, theoretically, it could continue to do so indefinitely. Additionally, because the newly synthesized DNA strand is covalently-bound to the original template, the displaced strand possesses the original genomic sequence (e.g., gene or other sequence of interest) at its 5′-terminus. In RCR, the original genomic sequence is followed by any number of “replication units” complementary to the original template sequence, wherein each replication unit is synthesized by continuing revolutions of said original template sequence. Hence, each subsequent revolution displaces the DNA which is synthesized in the previous replication cycle.

In vivo, RCR is utilized in several biological systems. For example, the genome of several bacteriophage are single-stranded, circular DNA. During replication, the circular DNA is initially converted to a duplex form, which is then replicated by the aforementioned rolling-circle replication mechanism. The displaced terminus generates a series of genomic units that can be cleaved and inserted into the phage particles. Additionally, the displaced single-strand of a rolling-circle can be converted to duplex DNA by synthesis of a complementary DNA strand. This synthesis can be used to generate the concatemeric duplex molecules required for the maturation of certain phage DNAs. For example, this provides the principle pathway by which λ bacteriophage matures. RCR is also used in vivo to generate amplified rDNA in Xenopus oocytes, and this fact may help explain why the amplified rDNA is comprised of a large number of identical repeating units. In this case, a single genomic repeating unit is converted into a rolling-circle. The displaced terminus is then converted into duplex DNA which is subsequently cleaved from the circle so that the two termini can be ligated together so as to generate the amplified circle of rDNA.

Through the use of the RCA reaction, a strand may be generated which represents many tandem copies of the complement to the circularized molecule. For example, RCA has recently been utilized to obtain an isothermal cascade amplification reaction of circularized padlock probes in vitro in order to detect single-copy genes in human genomic DNA samples (see Lizardi, et al., 1998. Nat. Genet. 19: 225-232). In addition, RCA has also been utilized to detect single DNA molecules in a solid phase-based assay, although difficulties arose when this technique was applied to in situ hybridization (see Lizardi, et al., 1998. Nat. Genet. 19: 225-232).

If desired, RCA can be performed at elevated temperatures, e.g., at temperatures greater than 37° C., 42° C., 45° C., 50° C., 60° C., or 70° C. RCA can be performed initially at a lower temperature, e.g., room temperature, and then shifted to an elevated temperature. Elevated temperature RCA is preferably performed with thermostable nucleic acid polymerases and with primers that can anneal stably and with specificity at elevated temperatures.

RCA can also be performed with non-naturally occurring oligonucleotides, e.g., peptide nucleic acids. Further, RCA can be performed in the presence of auxiliary proteins such as single-stranded binding proteins.

The development of a method of amplifying short DNA molecules which have been immobilized to a solid support, termed RCA has been recently described in the literature (see e.g., Hatch, et al., 1999. Genet. Anal. Biomol. Engineer. 15: 35-40; Zhang, et al., 1998. Gene 211: 277-85; Baner, et al., 1998. Nucl. Acids Res. 26: 5073-5078; Liu, et al., 1995. J. Am. Chem. Soc. 118: 1587-1594; Fire and Xu, 1995. Proc. Natl. Acad. Sci. USA 92: 4641-4645; Nilsson, et al., 1994. Science 265: 2085-2088). RCA targets specific DNA sequences through hybridization and a DNA ligase reaction. The circular product is then subsequently used as a template in a rolling circle replication reaction.

RCA driven by DNA polymerase can replicate circularized oligonucleotide probes with either linear or geometric kinetics under isothermal conditions. In the presence of two primers (one hybridizing to the + strand, and the other, to the − strand of DNA), a complex pattern of DNA strand displacement ensues which possesses the ability to generate 1×10⁹ or more copies of each circle in a short period of time (i.e., less-than 90 minutes), enabling the detection of single-point mutations within the human genome. Using a single primer, RCA generates hundreds of randomly-linked copies of a covalently closed circle in several minutes. If solid support matrix-associated, the DNA product remains bound at the site of synthesis, where it may be labeled, condensed, and imaged as a point light source. For example, linear oligonucleotide probes, which can generate RCA signals, have been bound covalently onto a glass surface. The color of the signal generated by these probes indicates the allele status of the target, depending upon the outcome of specific, target-directed ligation events. As RCA permits millions of individual probe molecules to be counted and sorted, it is particularly amenable for the analysis of rare somatic mutations. RCA also shows promise for the detection of padlock probes bound to single-copy genes in cytological preparations.

In addition, a solid-phase RCA methodology has also been developed to provide an effective method of detecting constituents within a solution. Initially, a recognition step is used to generate a complex h a circular template is bound to a surface. A polymerase enzyme is then used to amplify the bound complex. RCA uses small DNA probes that are amplified to provide an intense signal using detection methods, including the methods described in more detail below.

Other examples of isothermal amplification systems include, e.g., (i) self-sustaining, sequence replication (see e.g., Guatelli, et al., 1990. Proc. Natl. Acad. Sci. USA 87: 1874-1878), (ii) the Qβ replicase system (see e.g., Lizardi, et al., 1988. BioTechnology 6: 1197-1202), and (iii) nucleic acid sequence-based amplification (NASBA™; see Kievits, et al., 1991. J. Virol. Methods 35: 273-286).

XII. Methods for Determining the Nucleotide Sequence of the Amplified Product

Amplification of a nucleic acid template as described above results in multiple copies of a template nucleic acid sequence covalently linked to an anchor primer. In one embodiment, a region of the sequence product is determined by annealing a sequencing primer to a region of the template nucleic acid, and then contacting the sequencing primer with a DNA polymerase and a known nucleotide triphosphate, i.e., dATP, dCTP, dGTP, dTTP, or an analog of one of these nucleotides. The sequence can be determined by detecting a sequence reaction byproduct, as is described below.

The sequence primer can be any length or base composition, as long as it is capable of specifically annealing to a region of the amplified nucleic acid template. No particular structure for the sequencing primer is required so long as it is able to specifically prime a region on the amplified template nucleic acid. Preferably, the sequencing primer is complementary to a region of the template that is between the sequence to be characterized and the sequence is capable of hybridizing to the anchor primer. The sequencing primer is extended with the DNA polymerase to form a sequence product. The extension is performed in the presence of one or more types of nucleotide triphosphates, and if desired, auxiliary binding proteins.

Amplification of a nucleic acid template as described above results in multiple copies of a template nucleic acid sequence covalently linked to an anchor primer. In one embodiment, a region of the sequence product is determined by annealing a sequencing primer to a region of the template nucleic acid, and then contacting the sequencing primer with a DNA polymerase and a known nucleotide triphosphate, i.e., dATP, dCTP, dGTP, dTTP, or an analog of one of these nucleotides. The sequence can be determined by detecting the sequence reaction addition to the nascent DNA, as is described below.

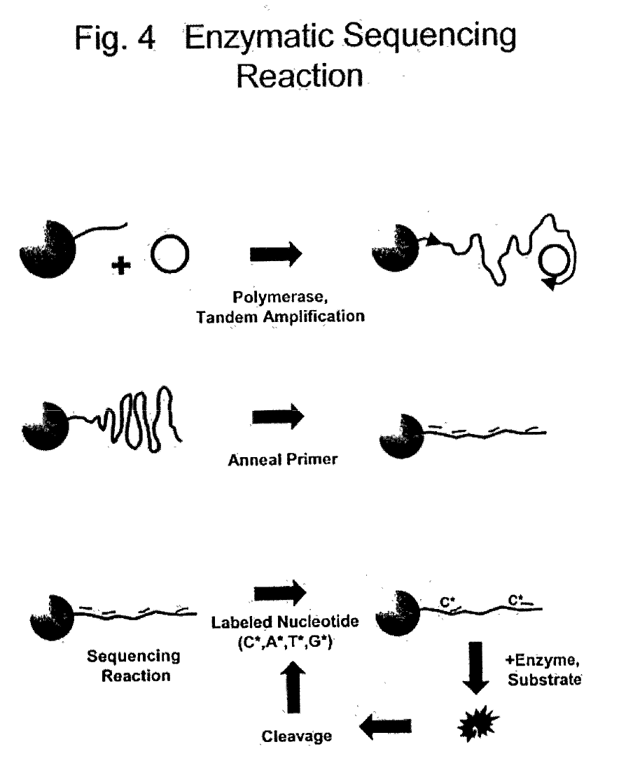

An example of one embodiment of the invention is shown in FIG. 4 where a circularized DNA fragment is annealed to a capture oligonucleotide immobilized a magnetic bead. The annealed circle is then tandem amplified and annealed with excess sequencing primers. The template is sequenced by enzymatic sequencing. Hapten-labeled nucleotide (for example biotin, digoxigenin), is first incorporated into the extending DNA strand by a DNA polymerase. An enzyme conjugated with a hapten-binding protein (SA-Enz) is then added to label the DNA. Chemiluminescent substrate for the enzyme (for example alkaline phosphatase, horseradish peroxidase, or β-galactosidase) is added to generate light (recorded by the detector). The labeling enzyme is then removed by reducing the disulfide bond by a reducing reagent (DTT, TCEP, glutathione, or β-mercaptoethanol). The cycle is repeated by adding a different dBTP-Hapten.

The sequence primer can be any length or base composition, as long as it is capable of specifically annealing to a region of the amplified nucleic acid template. No particular structure is required for the sequencing primer so long as it is able to specifically prime a region on the amplified template nucleic acid. Preferably, the sequencing primer is complementary to a region of the template that is between the sequence to be characterized and the sequence is capable of being hybridized to the anchor primer. The sequencing primer is extended with the DNA polymerase to form a sequence product. The extension is performed in the presence of one or more types of nucleotide triphosphates, and if desired, auxiliary binding proteins.

Incorporation of the dNTP is preferably determined by assaying for the presence of a hapten associated with the incorporated nucleotide. In a preferred embodiment, the nucleotide sequence of the anchored DNA is determined by measuring the presence of a biotin molecule linked via a disulfide to the specific dNTP. The presence of the biotin associated with the growing DNA chain is revealed via an enzyme-linked streptavidin molecule and a chemiluminescent substrate. Addition of the substrate and either subsequent absence or evolution of light identifies the nucleotide. The hapten is removed in this embodiment through the addition of a reducing agent. Such methods enable a nucleotide to be identified in a given target position, and the DNA to be sequenced simply and rapidly while avoiding the need for electrophoresis and the use of potentially dangerous radiolabels.

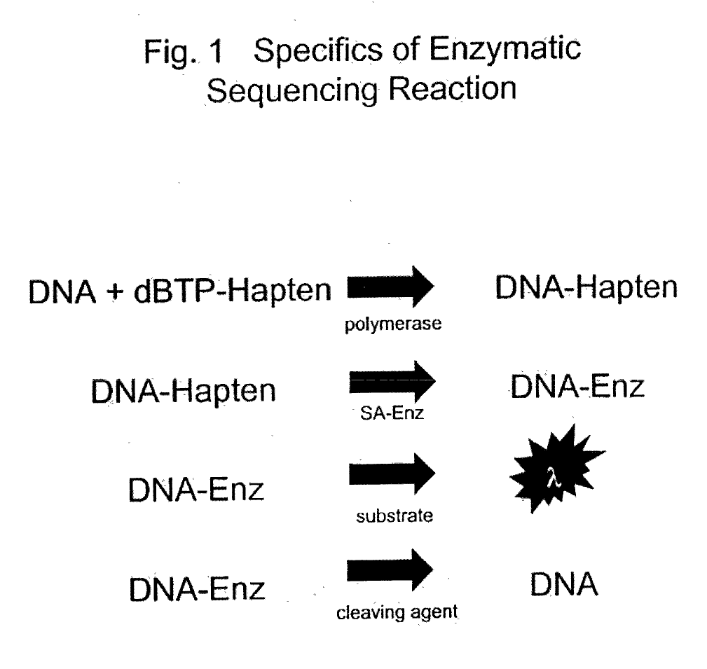

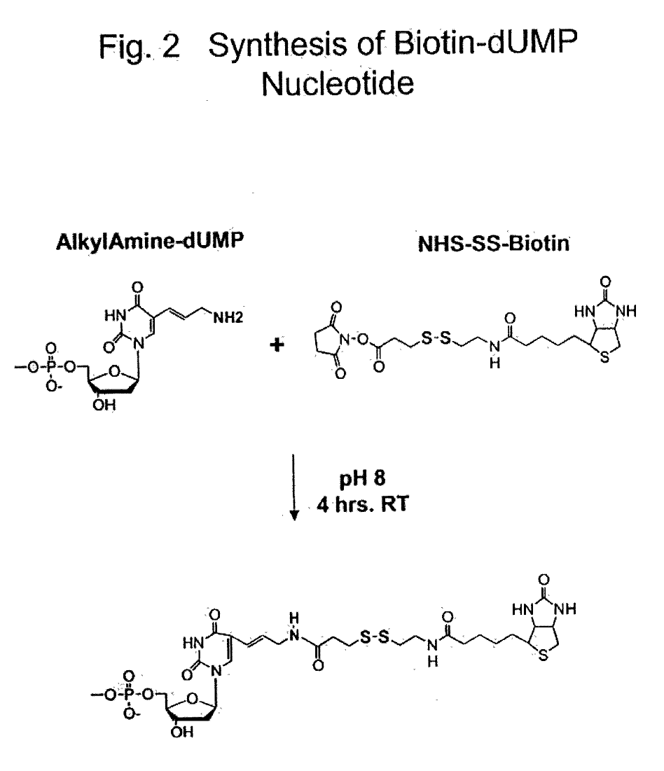

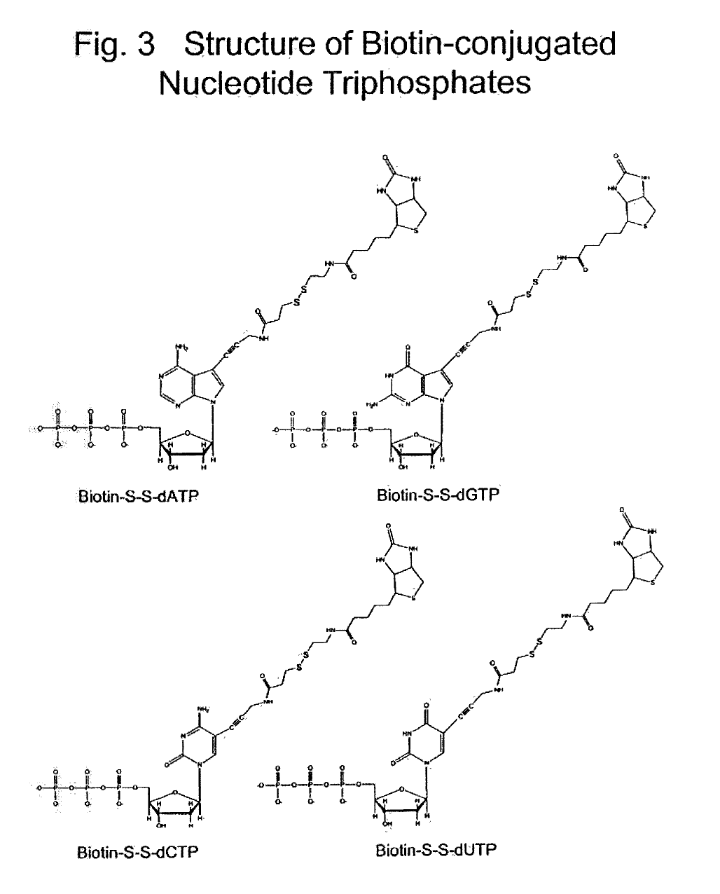

An example of the method just described in shown in FIG. 1. dBTP-Hapten is first incorporated into the extending DNA strand by a DNA polymerase. An enzyme conjugated with a hapten-binding protein (SA-Enz) is then added to label the DNA. Chemiluminescent substrate for the enzyme (for example, alkaline phosphatase, horseradish peroxidase, or β-galactosidase) is added to generate light (recorded by the detector). The labeling enzyme is then removed by reducing the disulfide bond by a reducing reagent (DTT, TCEP, glutathione, or β-mercaptoethanol). The cycle is repeated by adding a different dBTP-Hapten. The biotinylated nucleotides may be synthesized, for example, using the synthesis techniques outlined in FIG. 2. In FIG. 3, the structure of the final biotin-S-S-dNTP are shown.

Suitable enzymes for converting substrates into light include luciferases, for example, insect luciferases. Luciferases produce light as an end-product of catalysis. The best known light-emitting enzyme is that of the firefly, Photinus pyralis (Coleoptera). The corresponding gene has been cloned and expressed in bacteria (see for example, de Wet, et al., 1985. Proc. Natl. Acad. Sci. USA 80: 7870-7873) and plants (see for example, Ow, et al., 1986. Science 234: 856-859), as well as in insect (see for example, Jha, et al, 1990. FEBS Lett. 274: 24-26) and mammalian cells (see for example, de Wet, et al., 1987. Mol. Cell. Biol. 7: 725-7373; Keller, et al., 1987. Proc. Natl. Acad. Sci. USA 82: 3264-3268). In addition, a number of luciferase genes from the Jamaican click beetle, Pyroplorus plagiophihalamus (Coleoptera), have recently been cloned and partially characterized (see for example, Wood, et al., 1989. J. Biolumin. Chemilumin. 4: 289-301; Wood, et al., 1989. Science 244: 700-702). Distinct luciferases can sometimes produce light of different wavelengths, which may enable simultaneous monitoring of light emissions at different wavelengths. Accordingly, these aforementioned characteristics are unique, and add new dimensions with respect to the utilization of current reporter systems.

Firefly luciferase catalyzes bioluminescence in the presence of luciferin, adenosine 5′-triphosphate (ATP), magnesium ions, and oxygen, resulting in a quantum yield of 0.88 (see for example, McElroy and Selinger, 1960. Arch. Biochem. Biophys. 88: 136-145). The firefly luciferase bioluminescent reaction can be utilized as an assay for the detection of ATP with a detection limit of approximately 1×10⁻¹³ M (see for example, Leach, 1981. J. Appl. Biochem. 3: 473-517). In addition, the overall degree of sensitivity and convenience of the luciferase-mediated detection systems have created considerable interest in the development of firefly luciferase-based biosensors (see for example, Green and Kricka, 1984. Talanta 31: 173-176; Blum, et al., 1989. J. Biolumin. Chemilumin. 4: 543-550). Luciferase can hydrolyze dATP directly with concomitant release of a photon. This results in a false positive signal because the hydrolysis occurs independent of incorporation of the dATP into the extended sequencing primer. To avoid this problem, a dATP analog can be used which is incorporated into DNA, i.e., it is a substrate for a DNA polymerase, but is not a substrate for luciferase. One such analog is α-thio-dATP. Thus, use of α-thio-dATP avoids the spurious photon generation that can occur when dATP is hydrolyzed without being incorporated into a growing nucleic acid chain.

Enzymes for which there are commercially available chemiluminescent substrates include β galactosidase, alkaline phosphatase, neuraminidase, and horse-radish peroxidase. Synthesis of other enzyme substrates based on luminol or dioxetane-ring structures is well known to those skilled in the art.

Alkaline phosphatase is frequently conjugated to streptavidin, avidin, or antibodies to be used as secondary detection reagents. These detection reagents are widely used in a variety of applications including ELISAs (Meth. Mol. Biol. 32, 461(1994)), immonuhistochemistry (J. Clin. Microbiol. 19, 230(1984)), and Northern, Southern and Western blot techniques. Chromogenic substrates (such as BCIP, which yields a dark blue precipitate), fluorogenic phosphotase substrates, and chemiluminescent substrates are available. CDP-Star® and CSPD® (available from Applied Biosystems, Foster City, Calif.) chemiluminescent substrates for alkaline phosphatase let you detect alkaline phosphatase and alkaline phosphatase-labeled molecules with unparalleled sensitivity, speed, and ease. Chemiluminescent substrates exhibit high sensitivity in membrane-based applications such as Southern, northern, and western blotting. Both substrates can also be used in solution-based assays such as immunoassays, DNA probe assays, enzyme assays, and reporter gene assays. Maximum light levels are reached in approximately 10 minutes and glow emission persists for several hours. Solution assays require chemiluminescent enhancers. Membrane-based assays may also require them for increased light output and sensitivity.

NA-Star™ chemiluminescent substrate (Applied Biosystems) enables sensitive detection of neuraminidase activity. This substrate is a highly sensitive replacement for the widely used fluorogenic substrate, methylumbelliferyl N-acetylneuraminic acid. 1,2-Dioxetane chemiluminescence substrates enable extremely sensitive detection of biomolecules by producing visible light that is detected with film or instrumentation. Chemiluminescence substrates emit visible light upon enzyme-induced decomposition, providing low background luminescence coupled with high intensity light output.

Chemiluminescent substrates are available for horse-radish peroxidase from several manufacturers, including Alpha Diagnostic International, Inc. (San Antonio, Tex.). Their Nu-Glo substrate is provided as a stable two-component solution, and is a luminol-based solution. In the presence of hydrogen peroxide, HRP converts luminol to an exited state dianion that emits light on return to its ground state. The resulting signal can be measured by using a camera luminometer or X-ray films to provide a permanent record.

Using one or more of the above-described enzymes, the sequence primer is exposed to a polymerase and a known dNTP which is a reversibly labeled and optionally reversibly terminated. If the dNTP is incorporated onto the 3′ end of the primer sequence, the label is incorporated. For most applications it is desirable to wash away diffusible sequencing reagents, for example, unincorporated dNTPs, with a wash buffer. Any wash buffer used in other types of sequencing reactions, can be used.

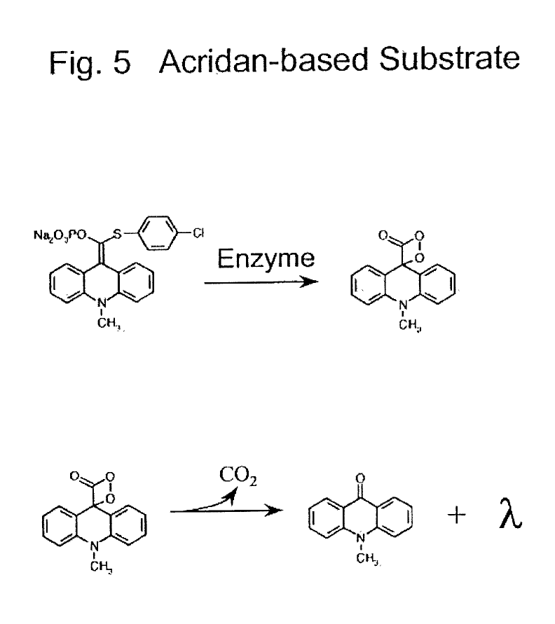

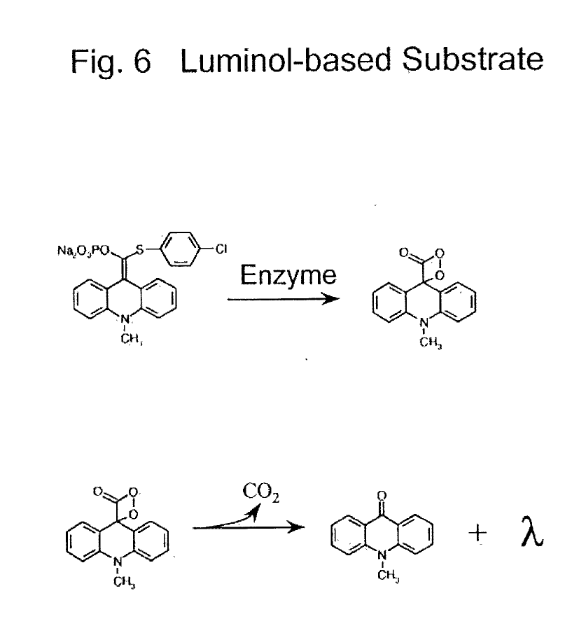

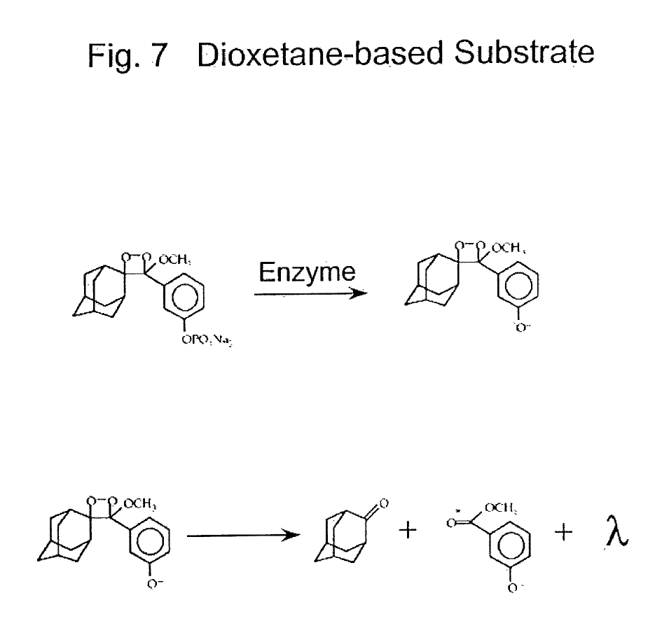

Other examples of substrates (labels) that can be detectable by emitted photons are shown in FIGS. 5-7. FIG. 5 shows the acridan-based substrates. Reaction of the acridan substrate with an enzyme results in an excited intermediate that can give off light. In the example diagrammed here, the reaction is between the Pierce Lumi-Phos WB substrate and alkaline phosphatase, though the enzyme used can vary depending on the cleavable moiety substituted onto the acridan molecule. FIG. 6 depicts the luminol-based substrates. Reaction of the luminol substrate with peroxidase results in an unstable intermediate that emits light and is converted into the 3-aminophtalate dianion. This is the reaction that occurs in the Pierce SuperSignal® ELISA Femto Maximum Sensitivity Substrate. FIG. 7 depicts the dioxetane based substrates. Reaction of the 1,2-dioxetane substrate with an enzyme results in an unstable intermediate that breaks apart to yield two product molecules, adamantanone and a chemically excited fluorophor, which can then give off light. In the example diagrammed here, the reaction is between Lumigen PPD and alkaline phosphatase. The enzyme used can vary depending on the cleavable moiety substituted onto the 1,2-dioxetane-based substrate.

In some embodiments, the concentration of reactants in the sequencing reaction include 1 pmol DNA, 3 pmol polymerase, 40 pmol dNTP in 0.2 ml buffer. The sequencing reaction can be performed with each of four predetermined nucleotides, if desired. A “complete” cycle generally includes sequentially administering sequencing reagents for each of the nucleotides dATP, dGTP, dCTP and dTTP (or dUTP), in a predetermined order. Unincorporated dNTPs are washed away between each of the nucleotide additions. Alternatively, unincorporated dNTPs are degraded by apyrase. The cycle is repeated as desired until the desired amount of sequence of the sequence product is obtained. In some embodiments, about 10-1000, 10-100, 10-75, 20-50, or about 30 nucleotides of sequence information is obtained from extension of one annealed sequencing primer.

If desired, apyrase may be “washed” or “flowed” over the surface of the solid support so as to facilitate the degradation of any remaining, non-incorporated dNTPs within the sequencing reaction mixture. Upon treatment with apyrase, any remaining reactants are washed away in preparation for the following dNTP incubation and photon detection steps. Alternatively, the apyrase may be bound to the solid support.

In another embodiment, a mixture of both unlabeled and labeled nucleotides are simultaneously extended onto a concatenated anchored DNA molecule. The enzyme-linked hapten-binding molecule is added and the appropriate substrate used as aforementioned to reveal the presence or absence of the specified nucleotide addition.

Alternatively, sequence byproducts can be generated using dideoxynucleotides having a label on the 3′ carbon. The label can be a hapten to which an enzyme-linked hapten-binding molecule that can be coupled with the aforementioned reactions can be used to reveal the presence or absence of nucleotide addition. In one embodiment, the label can be cleaved to reveal a 3′ hydroxyl group that can then serve as a substrate for subsequent nucleotide extensions. In this method, addition of a given nucleotide is scored as positive or negative, and one base is determined at each trial. In this embodiment, solid phase enzymes are not required and multiple measurements can be made.

Alternatively, sequence byproducts can be generated using a dideoxynucleotide having a label on a position other than the 3′ carbon. Again, the label can be a hapten to which an enzyme-linked hapten-binding molecule that can be coupled with the aforementioned reactions can be used to reveal the presence or absence of nucleotide extensions. In one embodiment, the modified dideoxynucleotide serves as a chain terminator. The use of a ratio of unmodified to labeled-dideoxynucleotide on extended anchored DNA at, for example, 1000 to 1, 100 to 1, 10 to 1, 1 to 1, 1 to 10, 1 to 100, 1 to 1000 allows for repeated DNA sequencing of various lengths, of a given signal intensity, dependent on the repeat number of amplified extended DNA. In this method, addition of a given nucleotide is scored as positive or negative, and one base is determined at each trial. In this embodiment, solid phase enzymes are not required and multiple measurements can be made.

The photons generated may be quantified using a variety of detection apparatuses, for example, a photomultiplier tube, charge-coupled display (CCD), CMOS, absorbance photometer, a luminometer, charge injection device (CID), or other solid state detector, as well as the apparatuses described herein. In a preferred embodiment, the quantitation of the emitted photons is accomplished by the use of a CCD camera fitted with a fused fiber optic bundle. In another preferred embodiment, the quantitation of the emitted photons is accomplished by the use of a CCD camera fitted with a microchannel plate intensifier. CCD detectors are described in, for example, Bronks, et al., 1995. Anal. Chem. 65: 2750-2757.

An exemplary CCD system is a Spectral Instruments, Inc. (Tucson, Ariz.) Series 600 4-port camera with a Lockheed-Martin LM485 CCD chip and a 1-1 fiber optic connector (bundle) with 6-8 um individual fiber diameters. This system has 4096×4096, or greater than 16 million, pixels and has a quantum efficiency ranging from 10% to >40%. Thus, depending on wavelength, as much as 40% of the photons imaged onto the CCD sensor are converted to detectable electrons.

For most applications it is desirable to use reagents free of contaminants like ATP and PPi. These contaminants may be removed by flowing the reagents through a pre-column containing apyrase and/-or pyrophosphatase bound to resin. Alternatively, the apyrase or pyrophosphatase can be bound to magnetic beads and used to remove contaminating ATP and PPi present in the reagents. In addition it is desirable to wash away diffusible sequencing reagents, e.g., unincorporated but reversibly labeled dNTPs, with a wash buffer. Any wash buffer used in sequencing can be used.

In some embodiments, the concentration of reactants in the sequencing reaction include 1 pmol DNA, 3 pmol polymerase, 40 pmol dNTP in 0.2 ml buffer. See Ronaghi, et al., Anal. Biochem. 242: 84-89 (1996).

The sequencing reaction can be performed with each of four predetermined nucleotides, if desired. A “complete” cycle generally includes sequentially administering sequencing reagents for each of the nucleotides dATP, dGTP, dCTP and dTTP (or dUTP), in a predetermined order. Unincorporated dNTPs are washed away between each of the nucleotide additions. Alternatively, unincorporated dNTPs are degraded by apyrase (see below). The cycle is repeated as desired until the desired amount of sequence of the sequence product is obtained. In some embodiments, about 10-1000, 10-100, 10-75, 20-50, or about 30 nucleotides of sequence information is obtained from extension of one annealed sequencing primer.

In some embodiments, the nucleotide is modified to contain a disulfide-derivative of a hapten such as biotin. The addition of the modified nucleotide to the nascent primer annealed to the anchored substrate is analyzed by a post-polymerization step that includes i) sequentially binding of, in the example where the modification is a biotin, an avidin- or streptavidin-conjugated moiety linked to an enzyme molecule, ii) the washing away of excess avidin- or streptavidin-linked enzyme, iii) the flow of a suitable enzyme substrate under conditions amenable to enzyme activity, and iv) the detection of enzyme substrate reaction product or products. The hapten is removed in this embodiment through the addition of a reducing agent. Such methods enable a nucleotide to be identified in a given target position, and the DNA to be sequenced simply and rapidly while avoiding the need for electrophoresis and the use of potentially dangerous radiolabels.

A preferred enzyme for detecting the hapten is horse-radish peroxidase. If desired, a wash buffer, can be used between the addition of various reactants herein. Apyrase can be used to remove unreacted dNTP used to extend the sequencing primer. The wash buffer can optionally include apyrase.

Haptens may be, for example, biotin, digoxygenin, the fluorescent dye molecules cy3 and cy5, and fluorescein, are incorporated at various efficiencies into extended DNA molecules. The attachment of the hapten can occur through linkages via the sugar, the base, and via the phosphate moiety on the nucleotide. Example means for signal amplification include fluorescent, electrochemical and enzymatic. In a preferred embodiment using enzymatic amplification, the enzyme, e.g. alkaline phosphatase (AP), horse-radish peroxidase (HRP), beta-galactosidase, luciferase, can include those for which light-generating substrates are known, and the means for detection of these light-generating (chemiluminescent) substrates can include a CCD camera.

In a preferred mode, the modified base is added, detection occurs, and the hapten-conjugated moiety is removed or inactivated by use of either a cleaving or inactivating agent. For example, if the cleavable-linker is a disulfide, then the cleaving agent can be a reducing agent, for example dithiothreitol (DTT), beta-mercaptoethanol, etc. Other embodiments of inactivation include heat, cold, chemical denaturants, surfactants, hydrophobic reagents, and suicide inhibitors.

Luciferase can hydrolyze dATP directly with concomitant release of a photon. This results in a false positive signal because the hydrolysis occurs independent of incorporation of the dATP into the extended sequencing primer. To avoid this problem, a dATP analog can be used which is incorporated into DNA, i.e., it is a substrate for a DNA polymerase, but is not a substrate for luciferase. One such analog is α-thio-dATP. Thus, use of α-thio-dATP avoids the spurious photon generation that can occur when dATP is hydrolyzed without being incorporated into a growing nucleic acid chain.

Typically, the detection is calibrated by the measurement of the light released as a result of the detectable label, following the addition of control nucleotides to the sequencing reaction mixture immediately after the addition of the sequencing primer. This allows for normalization of the reaction conditions. Incorporation of two or more identical nucleotides in succession is revealed by a corresponding increase in the amount of light released. Thus, a two-fold increase in released light relative to control nucleotides reveals the incorporation of two successive dNTPs into the extended primer.

When the support is planar, the sequencing reactions preferably take place in a thin reaction chamber that includes one optically transparent solid support surface and an optically transparent cover. In some embodiments, the array has a planar top surface and a planar bottom surface, the planar top surface has at least 1,000 cavities thereon each cavity forming a reaction chamber. In additional embodiments, the planar bottom surface is optically conductive such that optical signals from the reaction chambers can be detected through the bottom planar surface. In a preferred embodiment, the distance between the top surface and the bottom surface is no greater than 10 cm. Sequencing reagents may then be delivered by flowing them across the surface of the substrate. More preferably, the cavities contain reagents for analyzing a nucleic acid or protein. In an additional embodiment, the array has a second surface spaced apart from the planar array and in opposing contact therewith such that a flow chamber is formed over the array. When the support is not planar, the reagents may be delivered by dipping the solid support into baths of any given reagents.

In a preferred embodiment, an array can be used to carry out separate parallel common reactions in an aqueous environment. The array can have a substrate having at least 1,000 discrete reaction chambers containing a starting material that is capable of reacting with a reagent, each of the reaction chambers being dimensioned such that when one or more fluids containing at least one reagent is delivered into each reaction chamber, the diffusion time for the reagent to diffuse out of the well exceeds the time required for the starting material to react with the reagent to form a product. The reaction chambers can be formed by generating a plurality of cavities on the substrate. The plurality of cavities can be formed in the substrate via etching, molding or micromaching. The cavities can have a planar bottom or a concave bottom. In a preferred embodiment, the substrate is a fiber optic bundle. In an additional embodiment, the reaction chambers are formed by generating discrete patches on a planar surface. The patches can have a different surface chemistry than the surrounding planar surface.

In various embodiments, some components of the reaction are immobilized, while other components are provided in solution. For example, in some embodiments, the enzymes utilized in the sequencing reaction (e.g., polymerase, luciferase) may be immobilized if desired onto the solid support. Similarly, one or more or of the enzymes may be immobilized at the termini of a fiber optic reactor array. When luciferase is immobilized, it is preferably less than 50 μm from an anchored primer. Other components of the reaction, e.g., a polymerase (such as Klenow fragment), nucleic acid template, and nucleotides can be added by flowing, spraying, or rolling. In still further embodiments, one more of the reagents used in the sequencing reactions is delivered on beads.

In some embodiments, reagents are dispensed using an expandable, flexible membrane to dispense reagents and seal reactors on FORA surface during extension reactions. Reagents can be sprayed or rolled onto either the FORA surface or onto the flexible membrane. The flexible membrane could then be either rapidly expanded or physically moved into close proximity with the FORA thereby sealing the wells such that PPi would be unable to diffuse from well to well. Preferably, data acquisition takes place at a reasonable time after reaction initiation to allow maximal signal to generate.

A sequence in an extended anchor primer can also be identified using sequencing methods other than by detecting a sequence byproduct. For example, sequencing can be performed by measuring incorporation of labeled nucleotides or other nucleotide analogs. These methods can be used in conjunction with fluorescent or electrochemiluminescent-based methods.

Alternatively, sequence byproducts can be generated using dideoxynucleotides having a label on the 3′ carbon. Preferably, the label can be cleaved to reveal a 3′ hydroxyl group. In this method, addition of a given nucleotide is scored as positive or negative, and one base is determined at each trial. In this embodiment, solid phase enzymes are not required and multiple measurements can be made.

In another embodiment, the identity of the extended anchor primer product is determined using labeled deoxynucleotides. The labeled deoxynucleotides can be, e.g., fluorescent nucleotides. Preferably the fluorescent nucleotides can be detected following laser-irradiation. Preferably, the fluorescent label reversible. One method of having a reversible label is to use a fluorescent that is not stable for long periods of exposure. If desired, the fluorescent signal can be quenched, e.g., photobleached, to return signal to background levels prior to addition of the next base. A preferred electrochemiluminescent label is ruthenium-tris-bi-pyridyl.

In one embodiment, a single stranded circular nucleic acid is immobilized in the reaction chamber; preferably each reaction chamber has no more than one single stranded circular nucleic acid disposed therein. More preferably, a single stranded circular nucleic acid is immobilized on a mobile solid support disposed in the reaction chamber. In another embodiment, each single stranded circular nucleic acid contains at least 100 copies of a nucleic acid sequence, each copy covalently linked end to end.

The invention also comprises kits for use in methods of the invention which could include one or more of the following components: (a) a test specific primer which hybridizes to sample DNA so that the target position is directly adjacent to the 3′ end of the primer; (b) a polymerase; (c) detection enzyme means for identifying PPi release; (d) deoxynucleotides including, in place of dATP, a dATP analogue which is capable of acting as a substrate for a polymerase but incapable of acting as a substrate for a said PPi-detection enzyme; and (e) optionally dideoxynucleotides, optionally ddATP being replaced by a ddATP analogue which is capable of acting as a substrate for a polymerase but incapable of acting as a substrate for a said PPi-detection enzyme. If the kit is for use with initial PCR amplification then it could also include the following components: (i) a pair of primers for PCR, at least one primer having means permitting immobilization of said primer; (ii) a polymerase which is preferably heat stable, for example Taq1 polymerase; (iii) buffers for the PCR reaction; and (iv) deoxynucleotides. Where an enzyme label is used to evaluate PCR, the kit will advantageously contain a substrate for the enzyme and other components of a detection system.

The following examples are meant to illustrate, not limit, the invention. All references, patent applications and patents cited in this application, including U.S. Pat. No. 6,274,320, is hereby incorporated by reference in their entirety.

### EXAMPLE 1

Construction of Anchor Primers Linked to a Cavitated Terminus Fiber Optic Array

The termini of a thin wafer fiber optic array are cavitated by inserting the termini into acid as described by Healey et al., Anal. Chem. 69: 2213-2216 (1997).

A thin layer of a photoactivatable biotin analog is dried onto the cavitated surface as described in Hengsakul and Cass (Bioconjugate Chem. 7: 249-254, 1996) and exposed to white light through a mask to create defined pads, or areas of active biotin. Next, avidin is added and allowed to bind to the biotin. Biotinylated oligonucleotides are then added. The avidin has free biotin binding sites that can anchor biotinylated oligonucleotides through a biotin-avidin-biotin link.

The pads are approximately 10 μm on a side with a 100 μm spacing. Oligonucleotides are added so that approximately 37% of the pads include one anchored primer. On a 1 cm² surface are deposited 10,000 pads, yielding approximately 3700 pads with a single anchor primer.

### EXAMPLE 2

FORA Preparation.

Circularized sequencing templates were annealed to capture deoxyoligonucleotide immobilized on either Dynal M-280 (Dynal) or MPG beads (CPG) (bead concentration was 10 mg/ml). One μl of the template-annealed beads was diluted with 100 μl Templiphi reaction mix (Pharmacia). A fiber optic reactor array (FORA) was pre-blocked with Blocker Blotto (Pierce) overnight according to manufacturer's recommendation. Following the overnight blocking, the FORA was placed in the heating chamber with the etched side face-up. Three μl of the diluted beads were added to 7 μl of Templiphi reaction mix in a well and the FORA was placed on a heating-chamber surface. The beads were spun down to the etched FORA using a Beckman Allegra centrifuge (2,000 rpm, 5 min). An additional 20 μl of Templiphi reaction mix was added to the wells. The wells were sealed with a piece of Microseal (MJ Research) and then the heating-chamber was placed in an orbital shaker (50 rpm, 30° C., O/N) to allow tandem amplification (TA) to occur. After overnight TANDEM AMPLIFICATION, the wells were washed three times with TE containing 150 mM NaCl. One μl of sequencing primer (100 pmole per μl in TE) was added to 20 μl TE (+150 mM NaCl) in the well. The heating-chamber was heated to 80° C., and then cooled slowly to room temperature by unplugging the power cord. The wells were washed again with TE containing 150 mM NaCl (20 μl×3). The overnight tandem amplified FORA with sequencing primers annealed was then removed from the heating-chamber and washed in 50 ml phosphate buffered saline (PBS) with 0.1% Tween. The FORA thus prepared was ready for enzymatic amplification sequencing.

### EXAMPLE 3

Enzymatic Amplification Sequencing.

The FORA was placed in the flow chamber of the embodied instrument, and the flow chamber was attached to the faceplate of a CCD camera. The FORA was further blocked with Blocker Blotto by flowing the blocking reagent. through the flow chamber at the rate of 1 ml per minute for 45 seconds. Then the flow was paused for a 5 minutes. Followed by a flow rate of 1 ml per minute for 15 seconds. The FORA was washed with PBS+0.1 % Tween at the flow rate of 3 mls per minute for two minutes. The extension step was performed by flowing biotin-ss-dNTP (5 μM) and 100 Units per ml Klenow (prepared in 25 mM Tricine+5 mM magnesium acetate and 1 mg/ml BSA. Once again, the flow rate was 1 milliliter of solution per minute for 45 seconds; followed by a paused flow (no flow) for a five 5 minute incubation; followed by a resumed flow at 1 milliliter per minute for 15 seconds. The FORA was washed with PBS+0.1% Tween (3 ml/min, 2 min). Horseradish peroxidase conjugated to streptavidin (Pierce) prepared in PBS+0.1 % Tween (20 μg/ml) was flowed into the chamber (1 ml per minute for 45 seconds, 5 minutes with no flow, 1 ml per minute for 15 seconds). The FORA was then washed with PBS+0.1 % Tween under conditions described above. Chemiluminescent substrate (Supersignal ELISA Femto, Pierce) was flowed into the chamber at 1 ml per min for 2 min. The CCD camera was synchronized at the beginning of this step to acquire images at the rate of approximately one image per minute. The FORA was washed as previously described. Dithiothreitol (0.5 M in PBS+0.1% Tween) was flown into the chamber to reduce the disulfide bridge between the dNTP and biotin (1 min/ml, 1 min, flow was paused at 45 sec into the step for a 5 min incubation). The FORA slide was again washed with PBS+0.1 % Tween. The procedure was then repeated (from the extension step) for subsequent biotin-S—S-dNTP extensions.

### EXAMPLE 4

Annealing and Amplification of Members of a Circular Nucleic Acid Library

The 5′ biotinylated, 26-mer oligonucleotide probes were immobilized on streptavidin-coated MPG (Magnetic Pore Glass) beads at concentrations ranging from 1 to 10,000 probes per bead. A two-fold molar excess of circularized 88-mer oligonucleotides were annealed to the probes in 20 mM Tris-acetate, pH 7.5, 5 mM magnesium acetate, 0.5 mM EDTA by heating to 90° C., then cooling to 25° C. at a rate of 0.1° C. per second, after which the probes were washed twice with room temperature annealing buffer to remove unbound circles. Approximately 500 beads were loaded onto a 3 mm diameter circular area on the surface of the FORA by centrifugation at 2000×gravity for 7 minutes. Tandem amplification was initiated by mixing the complexed bead/primer/circle mixture with the reaction mixture (comprised of 33 mM Tris-acetate pH 7.9, 10 mM magnesium acetate, 66 mM potassium acetate, 0.1 mg/ml BSA, 0.4 mM dNTPS, 0.12% Tween) and incubating the FORA at 31° C. for 12 to 16 hours. Tandem amplification was halted through addition of excess dideoxy terminator deoxynucleotide triphosphates.

### EXAMPLE 5

Test of Enzyme Activity on Various Substrates.

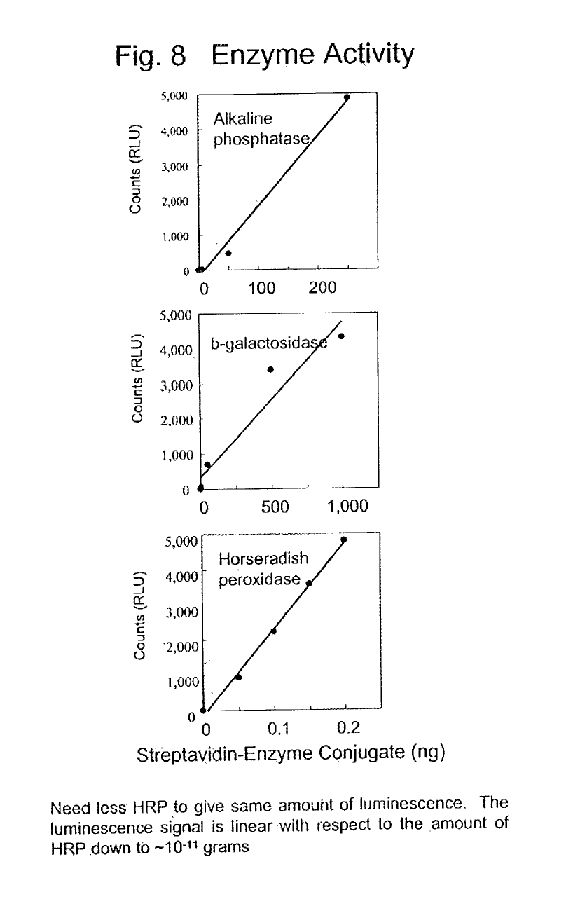

Three different enzymes were evaluated on a Turner TD 20/20 Luminometer (FIG. 8). For each enzyme, an optimal substrate was chosen based on the initiation time for the luminescence following addition of substrate to the enzyme, as well as on the amount of luminescence. The amount of luminescence for each enzyme after 90 seconds was plotted versus the amount of enzyme added, in order to determine the linearity of the luminescence signal with respect to the quantity of enzyme. Best results were obtained with horseradish peroxidase and the Pierce ELISA Femto Max Substrate. Linearity between enzyme and luminescence was observed with as little as 50 picograms of streptavidin-horseradish peroxidase conjugate.

### EXAMPLE 6

Output from a Sequenceing Reaction.

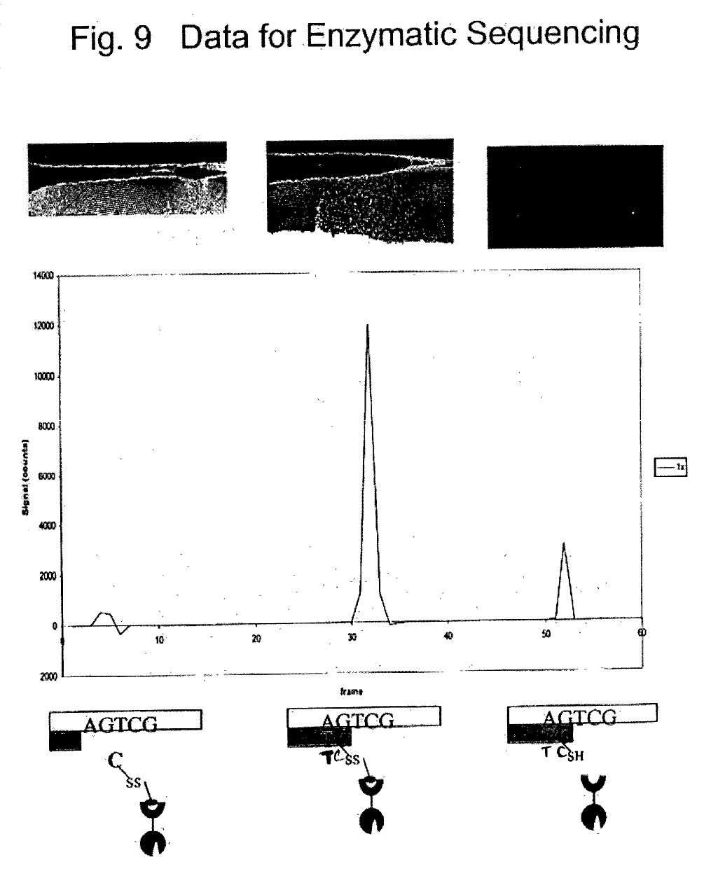

Results of enzymatic sequencing is shown in FIG. 9. The first peak (around frame 5) represents bright pixels. The second peak (frame 30) is indicative of a positive nucleotide addition (dC into dG template). Individual bright pixels can be seen, suggesting the DNA-immobilized beads were successfully labeled with horseradish peroxidase. The third peak indicates the remaining amount of labeled horseradish peroxidase after the reduction of the disulfide linkage by 0.5 mM DTT. The reduction efficiency is approximately 70%. Background was subtracted against negative control beads (i.e., beads without DNA immobilized).

### EXAMPLE 7

Optimization of Enzymatic Amplification

The general concept behind enzymatic amplification is simple. Primer is annealed to the DNA to be sequenced, and DNA polymerase adds the next nucleotide to the primer. The incorporated dNTP is a non-natural nucleotide, modified such that it is linked through a disulfide bond to a hapten. Next, the hapten is bound by an anti-hapten molecule conjugated to an enzyme that is capable of turning over chemilluminescent substrate. Substrate is added, and primers that had been extended with the hapten-labeled nucleotide produce light. Next, a reductant is added to cleave the disulfide bond between the nucleotide and the hapten, and the primer is ready for the next nucleotide addition.

The results of efforts to identify and optimize the reagents, enzymes, and conditions for enzymatic amplification are summarized below.

Nucleotide

Biotin is used as the nucleotide-labeling hapten, to take advantage of the strong interaction between biotin and avidin. Biotin-linked dNTP's were custom synthesized by NEN Life Sciences (division of Perkin Elmer). dNTP were labeled with both a 12-carbon linker between nucleotide and biotin (dNTP-12-BT) or with a 20-carbon linker between the nucleotide and the biotin (dNTP-20-BT). In the longer linkers, the disulfide bond was located in the linker at positions 8 and 9 from the nucleotide. The longer distance of the 20 carbon link between the biotin and the disulfide bond allowed for complete reduction of the disulfide bond with less rigorous conditions than for the dNTP-12-BT.

The two nucleotides could be compared by measuring the number of labeling enzyme molecules (horseradish peroxidase, HRP) bound per bead for each nucleotide. It was found from these experiments that the dNTP-20-BT resulted in about 30% less HRP/bead than dNTP-12-BT. The improved reduction of dNTP-20-BT (described in the paragraph above) cause it to be chosen for further experimentations.

It was found that an overall nucleotide concentration of 5 μM works well with the chosen polymerase (Sequenase), with a doping level of 50:50 biotinylated:normal nucleotides.

Polymerase

Different polymerases were tested for use with the biotinylated nucleotides. The key qualities desired in the polymerases were the ability to incorporate the non-natural nucleotides, and the correct incorporation of the nucleotides. Klenow readily incorporates the biotinylated nucleotides, but has a high rate of misincorporation. Other polymerases were tested, including Sequenase, BST polymerase, Phi 29, MMuLV, T4 Polymerase, Vent (exo-) and Taq polymerase, all at room temperature. Sequenase was chosen as the standard polymerase for biotinylated nucleotide incorporation on the basis of its low rate of misincorporation.

With Sequenase chosen, the conditions for optimal activity had to be determined. The Sequenase buffer provided by US Biochem for dideoxy sequencing was used as a basis, that is, 5 units Sequenase/ml, 5 mM MgCl₂, 50 mM NaCl, 20 mM Tris (pH 7.5), and 5 mg/ml BSA. A molecular crowding agent (polyvinyl pyrrolidone 360,000 MW, at 0.4 mg/ml) was added in order to drive the Sequenase onto the oligos.

From PPi generation, it was known that there were about 500,000 oligos/bead, however when the enzymatic amplification was carried out in PCR tubes for luminometer assays, it was found that only about 35,000 HRP were bound per bead. Furthermore, when enzymatic amplification was carried out on the Rig, only an estimated 4,000 HRP were bound per bead. These sequential losses in efficiency are not easy to explain. It is possible that since Sequenase is highly processive, after extending the primer, the polymerase might not release the DNA. In that case, the steric bulk of the polymerase might hinder streptavidin from binding to the biotinylated nucleotide, thereby lowering subsequent HRP binding efficiency. A number of wash conditions were tested for their efficacy in releasing Sequenase from the DNA strand, including high salt solutions to disrupt the ionic interaction between Sequenase and DNA, altered pH or detergent composition of the wash solution, 6 M guanidine-HCl, and a dideoxy-terminated DNA trap. None of these washes increased the number of HRP bound per bead significantly in luminometer assays. The more rigorous washes might have helped on the Rig, where the enzymatic amplification efficiency is so much lower than off the Rig. With this in mind, a 2.5 M NaCl, 0.1% Tween solution was used to wash the beads on the rig.

Most of the Rig and luminometer enzymatic amplification experiments were carried out at room temperature.

Labeling Enzymes

Several different binding and signal enzymes were tested for optimizing the sequencing signal. For binding to the biotinylated nucleotides, avidin, neutravidin, and streptavidin were tested. Streptavidin bound well to the biotinylated nucleotides, with minimal nonspecific binding on the FORA, so it was used for all subsequent experiments. Several signal enzymes were used as well. Alkaline phosphatase, Beta-galactosidase, horseradish peroxidase and luciferase were all tried at different stages of the project. Horseradish peroxidase proved to be the most sensitive enzyme for chemiluminescence, with sensitivities 100×, 500×, and 10,000× higher than for alkaline phosphatase, beta-galactosidase and luciferase, respectively. The lower limit of detection for HRP on the rig was approximately 2,000 HRP/bead.

There are several chemiluminescent substrates available for use with HRP. Among those tried, Pierce's ELISA FemtoMax proved to be the most sensitive, more so than Pierce's PicoMax substrate or Perkin Elmer's DNA Thunder substrate. Also important to consider was the rise time for the chemiluminescence from the different substrates. The signal from FemtoMax substrate typically rose to a maximum within one minute, whereas the other substrates took upwards of five minutes to peak.

There were several strategies for binding the HRP to the biotinylated nucleotides. The first used an HRP-streptavidin conjugate, with an average of two HRP molecules per streptavidin. This worked well, but binding streptavidin first, followed by biotinylated HRP in a second step resulted in about 50% more HRP bound per bead. The streptavidin-HRP conjugate was probably too bulky to bind efficiently to the biotinylated DNA. This was also suspected to be the case in a later experiment to bind Amdex dextran conjugates, in which up to 80 HRP molecules are bound to a dextran strand, along with 5-15 biotin molecules. The dextran conjugates were compared to biotinylated single HRP molecules for their ability to bind to beads that had already been extended with biotinylated nucleotides and bound with streptavidin. It was found that the Amdex conjugate had approximately 100× less HRP bound/bead than for the mono-HRP.

High background due to non-specific binding was a concern of using the FORA. It was found that streptavidin and biotinylated HRP at 5 nM each was the optimal concentration for both enzymes. Further, the FORA was treated with polyethylene glycol (PEG) to reduce background noise. Treatment may be by preincubation of the FORA with a solution of PEG.

Mobile Support (Beads)

DNA primers with a 5′ amine group were synthesized and chemically bound directly to epoxy beads, or through an EDAC intermediate to carboxyl-coated beads. 4.5 μM epoxy beads (Dynal) had the highest oligo load, typically binding 500K-800K oligos/bead. The loadings on carboxyl beads were much lower, around 100K oligos/bead.

For the purposes of enzymatic amplification, it was preferred to use a spacer in between the amine group and the primer DNA. The more preferred spacer tested was a chain of 24 adenines, followed by a C18 linker followed by the primer sequence. The C18 linker prevented the 3′ end of the oligo from being extended onto the 24A spacer.

Reductant

There are a number of different chemicals that could reduce the disulfide bond between the nucleotide and the biotin. Numerous reducing agents were tested but were found to be sub optimal. It was found that dithiothreitol (DTT) provided the most consistent results so it was chosen as the reductant for the enzymatic amplification experiments. Other reducing agents provided acceptable results but DTT was the optimal reducing agent. With dNTP-12-BT, it was found that the addition of 5% SDS to the reductant solution helped reduce the disulfide bond. Other detergents provided results but SDS was the optimal detergent.

Surprisingly, it was found that With the introduction of dNTP-20-BT, the SDS and DTT concentrations could be lowered while still resulting in ˜100% reduction. The final concentrations used were 50 mM DTT and 2.5% SDS.

We claim:

1. A reversibly labeled nucleotide comprising:
(a) a nucleotide or nucleoside,
(b) at least one detectable label comprising a light generating moiety which emits light in the presence of a substrate, and
(c) a linker connecting said nucleotide or nucleoside and said detectable label wherein said linker comprise a carbon chain of between 10 carbons to 24 carbons and a cleavable bond which can be cleaved to separate (a) from (b).

2. The reversible labeled nucleotide of claim 1 wherein said nucleotide is selected from the group consisting of a nucleotide monophosphate, a nucleotide diphosphate, and a nucleotide triphosphate.

3. The reversible labeled nucleotide of claim 1 wherein said nucleotide triphosphate is selected from the group consisting of dATP, dTTP, dGTP, dCTP, ATP, UTP, GTP and CTP.

4. The reversible labeled nucleotide of claim 1 wherein said light generating moiety is connected to said linker by a specific binding pair.

5. The reversible labeled nucleotide of claim 4 wherein said binding pair is biotin/avidin, biotin/streptavidin, disulfide derivatives or functional derivatives and analogs thereof.

6. The reversible labeled nucleotide of claim 5 wherein said disulfide derivative is a disulfide derivative of biotin.

7. The reversible labeled nucleotide of claim 4 wherein said binding pair is selected from the group consisting of antigen/antibody, hapten/peptide, maltose/maltose binding protein, protein A/antibody fragment, protein G/antibody fragment, polyhistidine/nickel, glutathione S transferase/glutathione and derivatives, functional fragments, and functional analogs thereof.

8. The reversible labeled nucleotide of claim 1 wherein said light generating moiety is selected from the group consisting of green fluorescent protein, blue fluorescent protein, red fluorescent protein, beta-galactosidase, chloramphenicol acetyltransferase, beta-glucoronidase, luciferases, b-lactamase, digoxygenin, and derivatives thereof.

9. The reversible labeled nucleotide of claim 8 wherein said fluorescent dye molecule is cy3 or cy5.

10. The reversible labeled nucleotide of claim 8 wherein said derivatives are selected from the group consisting of blue EBFP, cyan ECFP, yellow-green EYFP, destabilized GFP variants, stabilized GFP variants and fusion variants

11. The reversible labeled nucleotide of claim 1 wherein said light generating moiety is alkaline phosphatase or horse radish peroxidase.

12. The reversible labeled nucleotide of claim 1 wherein said substrate is selected from the group consisting of ATP, NBR/BCIP, ascorbate, ferrocyanide, cytochrome C X-gal, Acetyl CoA, n-butytyl CoA, chloramphenicol, glucoronides, antidigoxigenin-POD, diaminobenzidine, luciferin, beta-lactam, glucuronides, H₂O₂ and a combination thereof.

13. The reversible labeled nucleotide of claim 1 wherein said at least one detectable label is detectable using chemical or enzymatic methods.

14. The reversible labeled nucleotide of claim 1 wherein said linker comprises a carbon chain of between about 18 to about 22 carbons.

15. The reversible labeled nucleotide of claim 1 wherein said linker comprises a carbon chain of about 20 carbons.

16. The reversible labeled nucleotide of claim 1 wherein said linker is connected to a sugar, a base or a phosphate moiety on said nucleotide triphosphate.

17. The reversible labeled nucleotide of claim 1 wherein said linker is connected to said nucleotide triphosphate by a cleavable bond.

18. The reversible labeled nucleotide of claim 1 wherein said detectable moiety is selected from the group consisting of fluorescent dye molecule, fluorescein and a combination thereof.

19. The reversible labeled nucleotide of claim 1 wherein said cleavable bond is a covalent or ionic bond.

20. The reversible labeled nucleotide of claim 19 wherein said cleavable bond is cleavable by exposure to a reducing agent.

21. The reversible labeled nucleotide of claim 1 wherein said reducing agent is selected form the group consisting of dithiothreitol, β-mercaptoethanol.

22. The reversible labeled nucleotide of claim 1 wherein said cleavable bond is cleavable by exposure to heat, cold, chemical denaturants, surfactants, hydrophobic reagents, and suicide inhibitors.

23. The reversible labeled nucleotide of claim 1 wherein said detectable label can be inactivated by exposure to reducing agents, heat, cold, chemical denaturants, surfactants, hydrophobic reagents, and suicide inhibitors.

24. A method of determining the incorporation of a nucleotide into an elongating chain of a nucleic acid by:
(a) contacting a nucleic acid with a first species of a reversibly labeled nucleotide triphosphate according to claim 1;
(b) detecting incorporation of said first species of nucleotide triphosphate to said elongating chain of nucleic acid by detecting light emitted by said light generating moiety in the presence of a detection substrate.

25. The method of claim 24, further comprising the steps of:
(c) inactivating or detaching said label;
(d) repeating steps (a), (b) and (c) using a second species of nucleotide triphosphate of claim 1 wherein said first species and said second species are different.

26. The method of claim 25 further comprising the step of determining the sequence of the elongating nucleic acid by recording the order of nucleotide triphosphate used in step (a) and the results of step (b).

27. The method of claim 24 wherein said light generating moiety is selected from the group consisting of alkaline phosphatase, horse radish peroxidase, digoxygenin, fluorescent dye molecule, and fluorescein.

28. The method of claim 24 wherein said light generating moiety is selected from the group consisting of alkaline phosphatase, horse radish peroxidase, green fluorescent protein, blue fluorescent protein, red fluorescent protein, beta-galactosidase, chloramphenicol acetyltransferase, beta-glucoronidase, luciferases, b-lactamase and derivatives thereof.

29. The method of claim 24 wherein said detection substrate is selected from the group consisting of ATP, NBR/BCIP, ascorbate, ferrocyanide, cytochrome C X-gal, Acetyl CoA, n-butytyl CoA, chloramphenicol, glucoronides, antidigoxigenin-POD, diaminobenzidine, luciferin, beta-lactam, glucuronides, H₂O₂ and a combination thereof.

30. The method of claim 24 wherein said elongating nucleic acid is elongating along a template nucleic acid and wherein the sequence of the template nucleic acid is determined.

31. The method of claim 24 wherein said chain elongation reaction is a transcription reaction, a replication reaction, a reverse transcription reaction.

32. The method of claim 24 wherein said light emitted is nonstoichlometrc.

33. The method of claim 24 wherein said light emitted is greater than 1000 photons per nucleotide triphosphate incorporated.

34. The method of claim 24 wherein said light emitted is greater than 100 photons per nucleotide triphosphate incorporated.

35. The method of claim 24 wherein said light emitted is greater than 10 photons per nucleotide triphosphate incorporated.

36. A reversibly labeled nucleotide comprising:
(a) a nucleotide or nucleoside,
(b) at least one conjugatable moiety that comprises one part of a binding pair, and
(c) a linker connecting said nucleotide or nucleoside and said detectable label wherein said linker comprise a carbon chain of between 10 carbons to 24 carbons and a cleavable bond which can be cleaved to separate (a) from (b).

37. The reversible labeled nucleotide of claim 36 wherein said one part of a binding pair is selected from a group of binding pairs consisting of antigen/antibody, hapten/peptide, maltose/maltose binding protein, protein A/antibody fragment, protein G/antibody fragment, polyhistidine/nickel, glutathione S transferase/glutathione and derivatives, functional fragments, and functional analogs thereof.

38. The nucleotide of claim 36 or 37 further comprising a detectable label connected to a complementary part of said one part of a binding pair.

39. A method for sequencing a nucleic acid, the method comprising:
(a) providing one or more nucleic acid anchor primers;
(b) providing a plurality of single-stranded nucleic acid templates disposed within a plurality of cavities on a planar surface, each cavity forming an analyte reaction chamber, wherein the reaction chambers have a center to center spacing of between 5 to 200 μm;
(c) annealing an effective amount of the nucleic acid anchor primer to at least one of the single-stranded templates to yield a primed anchor primer-template complex;
(d) combining the primed anchor primer-template complex with a polymerase to form an extended anchor primer covalently linked to multiple copies of a nucleic acid complementary to the nucleic acid template;
(e) annealing an effective amount of a sequencing primer to one or more copies of said covalently linked complementary nucleic acid;
(f) extending the sequencing primer with a polymerase and a predetermined reversibly labeled nucleotide triphosphate according to claim 36 or 38 to yield a sequencing product; and
(g) detecting the amount of incorporation of said reversibly labeled triphosphate, thereby determining the sequence of the nucleic acid.

40. The method of claim 39 wherein said detecting step comprises the steps of
(a) contacting said nucleic acid with a detectable label a detectable label connected to a complementary part of said one part of a binding pair;
(b) detecting the incorporation of said detectable label to said extended sequencing primer.

41. The method of claim 39 or 40 further comprising the step of removing said detectable label after said detecting step.

42. The method of claim 41 wherein said step of removing said detectable label comprises exposing said label to a reducing agent.

43. The method of claim 42 wherein said reducing agent is selected form the group consisting of dithiothreitol, β-mercaptoethanol.

44. The method of claim 42 wherein said step of removing comprise exposing said label to heat, cold, chemical denaturants, surfactants, hydrophobic reagents, and suicide inhibitors.

45. The method of claim 39 or 40 further comprising the step of inactivating said detectable label.

46. The method of claim 45 wherein said step of inactivating said detectable label comprise exposing said label to heat, cold, chemical denaturants, surfactants, hydrophobic reagents, and suicide inhibitors.

47. The method of claim 39 wherein each single stranded nucleic acid is circular.

48. The method of claim 39 wherein each single stranded circular nucleic acid contains at least 100 copies of a nucleic acid sequence, each copy covalently linked end to end.

49. The method of claim 39 wherein each reaction chamber has a width in at least one dimension of between 0.3 μm and 100 μm.

50. The method of claim 39 wherein each reaction chamber has a width in at least one dimension of between 0.3 μm and 20 μm.

51. The method of claim 39 wherein each reaction chamber has a width in at least one dimension of between 0.3 μm and 10 μm.

52. The method of claim 39 wherein each reaction chamber has a width in at least one dimension of between 20 μm and 70 μm.

53. The method of claim 39 wherein the cavities number greater than 400,000.

54. The method of claim 39 wherein the cavities number between 400,000 and 20,000,000.

55. The method of claim 39 wherein the cavities number between 1,000,000 and 16,000,000.

56. The method of claim 39 wherein the center to center spacing is between 10 to 150 μm.

57. The method of claim 39 wherein the center to center spacing is between 50 to 100 μm.

58. The method of claim 39, wherein each cavity has a depth of between 10 μm and 100 μm.

59. The method of claim 39 wherein each cavity has a depth that is between 0.25 and 5 times the size of the width of the cavity.

60. The method of claim 39 wherein each cavity has a depth that is between 0.3 and 1 times the size of the width of the cavity.

61. The method of claim 39 wherein the nucleic acid sequence is further amplified to produce multiple copies of said nucleic acid sequence after being disposed in the reaction chamber.

62. The method of claim 61 wherein the nucleic acid sequence is amplified using an amplification technology selected from the group consisting of polymerase chain reaction, ligase chain reaction and isothermal DNA amplification.

63. The method of claim 39 wherein the single stranded nucleic acid is immobilized in the reaction chamber.

64. The method of claim 39 wherein the single stranded nucleic acid is immobilized on one or more mobile solid supports disposed in the reaction chamber.

65. A method for sequencing a nucleic acid, the method comprising:
(a) providing at least one nucleic acid anchor primer;
(b) providing a plurality of single-stranded circular nucleic acid templates in an array having at least 400,000 discrete reaction sites;
(c) annealing a first amount of the nucleic acid anchor primer to at least one of the single-stranded circular templates to yield a primed anchor primer-circular template complex;
(d) combining the primed anchor primer-circular template complex with a polymerase to form an extended anchor primer covalently linked to multiple copies of a nucleic acid complementary to the circular nucleic acid template;
(e) annealing a second amount of a sequencing primer to one or more copies of the covalently linked complementary nucleic acid;
(f) extending the sequencing primer with a polymerase and a predetermined nucleotide triphosphate according to claim 36 or 38 to yield a sequencing product and, when the predetermined nucleotide triphosphate is incorporated onto the 3′ end of the sequencing primer; and
(g) identifying the detectable label, thereby determining the sequence of the nucleic acid at each reaction site that contains a nucleic acid template.

66. The method of claim 65 further comprising the step of:
(h) removing or inactivating said detectable label.

67. The method of claim 66 further comprising the step of repeating steps (f) (g) and (h) with a different labeled nucleotide triphosphate.

68. The method of claim 65, wherein the anchor primer is linked to a particle.

69. The method of claim 68, wherein the anchor primer is linked to the particle prior to formation of the extended anchor primer.

70. The method of claim 68, wherein the anchor primer is linked to the particle after formation of the extended anchor primer.

71. A method of determining the base sequence of a plurality of nucleotides on an array, the method comprising:
(a) providing a plurality of sample DNAs, each disposed within a plurality of cavities on a planar surface, each cavity forming an analyte reaction chamber, wherein the reaction chambers have a center to center spacing of between 5 to 200 μm,
(b) adding a nucleotide 5′-triphosphate precursor according to claim 38 or 39, wherein said nucleotide is of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the nucleoside 5′-triphosphate precursor onto the 3′-end of the primer strands, provided the nitrogenous base of the nucleoside 5′-triphosphate precursor is complementary to the nitrogenous base of the unpaired nucleotide residue of the templates;
(c) detecting the incorporation of the reversible label to determine whether or not nucleoside 5′-triphosphate precursor was incorporated into the primer strands indicating that the unpaired nucleotide residue of the template has a nitrogenous base composition that is complementary to that of the incorporated nucleoside 5′-triphosphate precursor;
(d) removing or inactivating said reversible label; and
(e) sequentially repeating steps (b), (c) and (d), wherein each sequential repetition adds and, detects the incorporation of said one type of a reversibly labeled nucleotide precursor of known nitrogenous base composition; and
(f) determining the base sequence of the unpaired nucleotide residues of the template in each reaction chamber from the sequence of incorporation of said nucleoside precursors.

72. The method of claim 71 further comprising the step of removing said reversible termination before or after step (d).

73. A method for determining the nucleic acid sequence in a template nucleic acid polymer, comprising:
(a) introducing a plurality of template nucleic acid polymers into a plurality of cavities on a planar surface, each cavity forming an analyte reaction chamber, wherein the reaction chambers have a center to center spacing of between 5 to 200 μm, each reaction chamber having a polymerization environment in which the nucleic acid polymer will act as a template polymer for the synthesis of a complementary nucleic acid polymer when nucleotides are added;
(b) successively providing to the polymerization environment a series of feedstocks, each feedstock comprising a reversibly labeled nucleotide of claim 36 or 38 selected from among the nucleotides from which the complementary nucleic acid polymer will be formed, such that if the nucleotide in the feedstock is complementary to the next nucleotide in the template polymer to be sequenced said reversibly labeled nucleotide will be incorporated into the complementary polymer;
(c) detecting the incorporation of said label to determine the identify of each nucleotide in the complementary polymer and thus the sequence of the template polymer.
